(19) United States
(12) Patent Application Publication
(10) Pub. No.: US 2026/0290549 A1
(43) Pub. Date: Sep. 24, 2026

(54) SYSTEM

(71) Applicant: SoftBank Group Corp., Tokyo (JP)

(72) Inventor: Yasutaka YUDA, Tokyo (JP)

(73) Assignee: SoftBank Group Corp., Tokyo (JP)

(21) Appl. No.: 19/568,864

(22) Filed: Mar. 17, 2026

(30) Foreign Application Priority Data
Mar. 19, 2025 (JP) 2025-045257

(51) Int. Cl. G16H 20/60; G06V 20/68

(52) U.S. Cl. CPC G16H 20/60; G06V 20/68

(57) ABSTRACT

A system includes a processor that is configured to receive, via an interface for inputting conditions from a user, one or more conditions from the user, acquire ingredient information inside a refrigerator by using one or more sensors, and generate a prompt that instructs a generative AI model to generate a menu based on the acquired ingredient information and the one or more conditions from the user.

### CROSS-REFERENCE TO RELATED APPLICATION

This application is based on and claims priority under 35 USC 119 from Japanese Patent Application No. 2025-045257 filed on Mar. 19, 2025, the disclosure of which is incorporated by reference herein.

### BACKGROUND

### Technical Field

The present disclosure relates to a system.

### Related Art

Japanese Patent Application Laid-Open (JP-A) No. 2022-180282 discloses a persona chatbot control method executed by at least one processor. The method includes steps of: receiving a user utterance, adding the user utterance to a prompt including a description of a chatbot character and an associated instruction sentence, encoding the prompt, and inputting the encoded prompt to a language model to generate a chatbot utterance responding to the user utterance.

Conventional menu recommendation systems mainly rely on static recipe databases and manual user input of available ingredients, and therefore cannot accurately reflect the actual contents of a user's refrigerator. As a result, such systems frequently propose menus that require additional purchases or ignore ingredients already present, which can lead to increased food waste and higher household costs. Furthermore, these systems often fail to sufficiently consider the user's health-related preferences, such as a desire for healthy or nutritionally balanced meals, and therefore cannot adequately support health-conscious menu planning. In addition, existing approaches generally do not take into account expiration dates of ingredients or the operational state of the refrigerator, and thus do not contribute to reducing food loss or optimizing electric power consumption. Accordingly, there is a need for a system that can: (i) automatically acquire ingredient information inside a refrigerator via sensors; (ii) generate, in cooperation with a generative AI model, menus that reflect both the user's conditions and the actual ingredient inventory; (iii) incorporate health-related conditions into the menu generation; and (iv) reduce food waste and optimize electric power consumption so as to provide an economic benefit to the user.

### SUMMARY

In order to solve the above-described problems, an embodiment of the present invention provides a system comprising a processor, wherein the processor is configured to receive, via an interface for inputting conditions from a user, one or more conditions from the user, acquire ingredient information inside a refrigerator by using one or more sensors, and generate a prompt that instructs a generative AI model to generate a menu based on the acquired ingredient information and the one or more conditions from the user. In one aspect, the processor is configured to reflect, in the prompt, one or more health-related conditions received from the user, and to generate a prompt that instructs the generative AI model to generate a healthy menu, thereby enabling menu proposals aligned with the user's health preferences. In another aspect, the processor is configured to take into account an expiration date of ingredients inside the refrigerator so as to reduce food waste, and to optimize electric power consumption so as to provide an economic benefit to the user, for example by promoting the use of ingredients nearing expiration and by coordinating menu generation with refrigerator operation.

The term “processor” refers to any hardware, circuitry, or combination of hardware and software, including but not limited to a central processing unit (CPU), microcontroller, digital signal processor (DSP), application-specific integrated circuit (ASIC), field-programmable gate array (FPGA), or system-on-chip (SoC), that is capable of executing instructions to perform the functions described herein.

The term “interface for inputting conditions from a user” refers to any hardware and/or software component that allows a user to provide one or more conditions to the system, including but not limited to a graphical user interface, touch screen, keyboard, mouse, microphone, voice recognition interface, or network-based input interface provided by a terminal such as a smartphone or personal computer.

The term “condition” refers to any parameter, preference, constraint, or requirement specified by the user that is used by the system in connection with menu generation, including but not limited to health-related conditions, desired cooking time, target number of servings, dietary restrictions, taste preferences, budget constraints, and ingredient inclusion or exclusion preferences.

The term “ingredient information” refers to data representing one or more ingredients present inside a refrigerator, including but not limited to ingredient type, name, quantity, unit, packaging state, storage location, and optionally metadata such as purchase date or expiration date.

The term “refrigerator” refers to any cooling appliance or storage device that maintains an interior space at a temperature suitable for storing food or ingredients, including but not limited to household refrigerators, commercial refrigerators, smart refrigerators equipped with communication functions, and refrigerator-freezer combinations.

The term “sensor” refers to any device or combination of devices that is capable of detecting, measuring, or identifying an object or condition inside the refrigerator, including but not limited to cameras, image sensors, weight sensors, temperature sensors, humidity sensors, RFID readers, barcode readers, and optical or electromagnetic sensors.

The term “generative AI model” refers to any artificial intelligence model or machine learning model that is configured to generate output content, such as text, natural language descriptions, or structured data representing a menu or recipe, based on an input prompt, including but not limited to large language models, transformer-based models, and other neural network models trained on recipe, cooking, or related data.

The term “prompt” refers to data supplied to the generative AI model as an input instruction or query, which may include natural language text, structured parameters, or a combination thereof, and that specifies, directly or indirectly, conditions, ingredient information, and other constraints to cause the generative AI model to generate a menu that satisfies the specified requirements.

The term “menu” refers to one or more proposed dishes, meals, or recipes, including information such as dish names, lists of required ingredients, quantities, preparation steps, cooking times, and optionally nutritional information, that is generated by or with the assistance of the generative AI model based on the prompt.

The term “health-related condition” refers to any user-specified condition concerning health, nutrition, or dietary requirements, including but not limited to preferences for low-calorie, low-salt, low-fat, high-protein, balanced nutrition, allergy avoidance, specific medical or dietary restrictions, and other conditions aimed at supporting a healthy diet.

The term “expiration date” refers to any time-related data associated with an ingredient that indicates a recommended use-by date, best-before date, or shelf-life limit, including explicit dates printed on packaging, inferred dates based on purchase date and typical shelf life, or system-calculated dates used to determine ingredient freshness.

The term “food waste” refers to ingredients or food items that remain unused until they pass their expiration date or otherwise become unsuitable for consumption and are therefore discarded, including partial or entire quantities of ingredients stored in the refrigerator.

The term “electric power consumption” refers to the amount of electrical energy used by the refrigerator and any associated devices or components of the system, including but not limited to compressors, lighting, sensors, communication modules, and processing units involved in the operation of the system.

The term “economic benefit” refers to a reduction in costs or an improvement in economic efficiency for the user, including but not limited to savings achieved by reduced food waste, optimized use of ingredients before expiration, and lower electric power consumption resulting from improved control or usage patterns of the refrigerator and related devices.

### BRIEF DESCRIPTION OF THE DRAWINGS

Exemplary embodiments of the present disclosure will be described in detail based on the following figures, wherein:

FIG. 1 is a schematic diagram illustrating an example of a configuration of a data processing system according to a first exemplary embodiment;

FIG. 2 is a schematic diagram illustrating an example of relevant functions of a data processing device and a smart device according to the first exemplary embodiment;

FIG. 3 is a schematic diagram illustrating an example of a configuration of a data processing system according to a second exemplary embodiment;

FIG. 4 is a schematic diagram illustrating an example of relevant functions of a data processing device and smart glasses according to the second exemplary embodiment;

FIG. 5 is a schematic diagram illustrating an example of a configuration of a data processing system according to a third exemplary embodiment;

FIG. 6 is a schematic diagram illustrating an example of relevant functions of a data processing device and a headset-type terminal according to the third exemplary embodiment;

FIG. 7 is a schematic diagram illustrating an example of a configuration of a data processing system according to a fourth exemplary embodiment;

FIG. 8 is a schematic diagram illustrating an example of relevant functions of a data processing device and a robot according to the fourth exemplary embodiment;

FIG. 9 illustrates an emotion map mapping plural emotions;

FIG. 10 illustrates an emotion map mapping plural emotions;

FIG. 11 is a sequence diagram showing the flow of data processing system processing in Example 1;

FIG. 12 is a sequence diagram showing the flow of data processing system processing in Application Example 1;

FIG. 13 is a sequence diagram showing the flow of data processing system processing in Example 2; and

FIG. 14 is a sequence diagram showing the flow of data processing system processing in Application Example 2.

### DETAILED DESCRIPTION

Description follows regarding an example of exemplary embodiments of a system according to technology disclosed herein, with reference to the appended drawings.

First, explanation follows regarding terminology employed in the following description.

In the following exemplary embodiments, a reference-numeral-appended processor (hereinafter simply referred to as “processor”) may be implemented by a single computation unit, and may be implemented by a combination of plural computation units. The processor may be implemented by a single type of computation unit, or may be implemented by a combination of plural types of computation units. Examples of computation unit include a central processing unit (CPU), a graphics processing unit (GPU), a general-purpose computing on graphics processing units (GPGPU), an accelerated processing unit (APU), and the like.

In the following exemplary embodiments, random access memory (RAM) appended with a reference numeral is memory temporarily stored with information, and is employed as working memory by a processor.

In the following exemplary embodiments, reference-numeral-appended storage is a single or plural non-volatile storage devices for storing various programs and various parameters and the like. Examples of non-volatile storage devices include flash memory (such as a solid state drive (SSD)), a magnetic disk (for example, a hard disk), magnetic tape, and the like.

In the following exemplary embodiments, a reference-numeral-appended communication interface (I/F) is an interface including a communication processor and an antenna or the like. The communication I/F has the role of communicating between plural computers. An example of a communication standard applied for the communication I/F is a wireless communication standard, such as a Fifth Generation Mobile Communication System (5G), Wi-Fi (registered trademark), Bluetooth (registered trademark), and the like.

In the following exemplary embodiments “A and/or B” has the same definition as “at least one out of A or B”. Namely, “A and/or B” may mean A alone, may mean B alone, or may mean a combination of A and B. Moreover, similar logic to “A and/or B” is applied when “and/or” is employed to link three or more items in the present specification.

### First Exemplary Embodiment

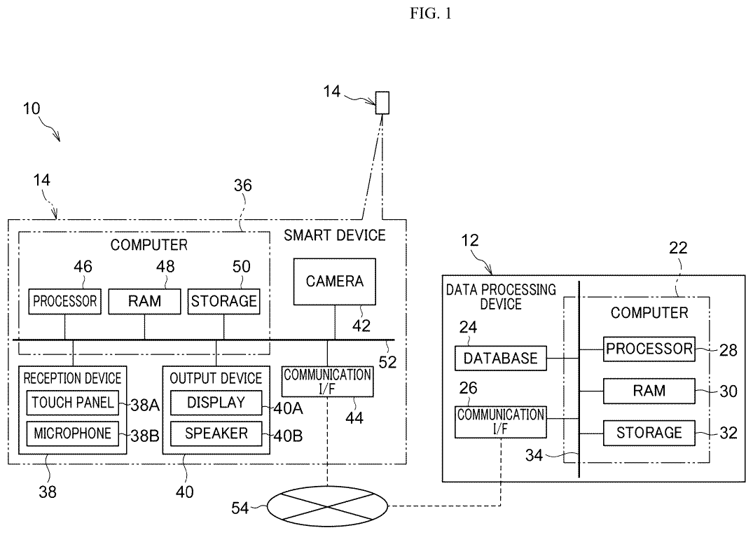

FIG. 1 illustrates an example of a configuration of a data processing system 10 according to a first exemplary embodiment.

As illustrated in FIG. 1, the data processing system 10 includes a data processing device 12 and a smart device 14. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart device 14 includes a computer 36, a reception device 38, an output device 40, a camera 42, and a communication I/F 44. The computer 36 includes a processor 46, RAM 48, and storage 50. The processor 46, the RAM 48, and the storage 50 are connected to a bus 52. The reception device 38, the output device 40, the camera 42, and the communication I/F 44 are also connected to the bus 52.

The reception device 38 includes a touch panel 38A, a microphone 38B, and the like for receiving user input. The touch panel 38A receives user input from contact of a pointer (for example, a pen, a finger, or the like) by detecting contact of the pointer. The microphone 38B receives spoken user input by detecting speech of the user. A control unit 46A in the processor 46 transmits data representing the user input received by the touch panel 38A and the microphone 38B to the data processing device 12. A specific processing unit 290 in the data processing device 12 acquires the data indicating the user input.

The output device 40 includes a display 40A, a speaker 40B, and the like for presenting data to a user 20 by outputting the data in an expression format perceivable by the user 20 (for example, audio and/or text). The display 40A displays visual information such as text, images, or the like under instruction from the processor 46. The speaker 40B outputs audio under instruction from the processor 46. The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like.

The communication I/F 44 is connected to the network 54. The communication I/F 44 and the communication I/F 26 perform the role of exchanging various information between the processor 46 and the processor 28 over the network 54.

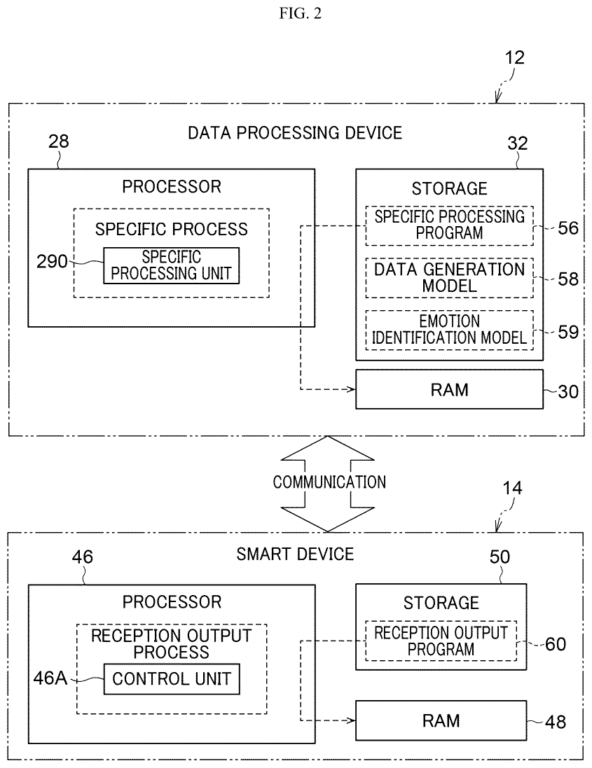

FIG. 2 illustrates an example of relevant functions of the data processing device 12 and the smart device 14.

As illustrated in FIG. 2, specific processing is performed by the processor 28 in the data processing device 12. A specific processing program 56 is stored in the storage 32. The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

A data generation model 58 and an emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart device 14. A reception and output program 60 is stored in the storage 50. The reception and output program 60 is employed by the data processing system 10 in combination with the specific processing program 56. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48. Note that a configuration may be adopted in which a similar data generation model and emotion identification model to the data generation model 58 and the emotion identification model 59 are included in the smart device 14, and these models are used to perform similar processing to the specific processing unit 290. The reception and output program is implemented by the processor 46 operating as the control unit 46A according to the reception and output program 60 executed in the RAM 48.

Note that devices other than the data processing device 12 may include the data generation model 58. For example, a server device (for example, a generation server) may include the data generation model 58. In such cases, the data processing device 12 performs communication with the server device including the data generation model 58 to obtain a processing result (prediction result or the like) obtained using the data generation model 58. The data processing device 12 may be a server device, and may be a terminal device owned by the user (for example, a mobile phone, a robot, a home electrical appliance, or the like). Next, description follows regarding an example of processing by the data processing system 10 according to the first exemplary embodiment.

### Example 1

Description follows regarding a flow of the specific processing in an Example 1. The units of the system described below are implemented by the data processing device 12 and the smart device 14. The data processing device 12 is called a “server” and the smart device 14 is called a “terminal”.

Conventional computer-implemented recipe and menu recommendation systems typically rely on fixed rule-based logic or simple filtering over static recipe databases. In such systems, a processor generally selects recipes by matching user-specified conditions, such as cooking time or dietary labels, against pre-stored metadata. These approaches have multiple technical drawbacks from the perspective of computer technology.

First, conventional systems do not efficiently exploit rich, unstructured preference data accumulated over time for each user. Even when past selection logs exist in storage, processors in known systems often treat these logs merely as counters or basic filters, and do not dynamically convert the logs into detailed, context-aware instructions for downstream computation. As a result, the interaction between user preference data in storage and the computation performed by downstream models is weak, leading to suboptimal use of memory and processing resources, and forcing repeated trial-and-error requests by the user.

Second, even where a generative AI model is introduced, conventional systems typically pass the user conditions almost verbatim as input text to the model. In such configurations, the processor does not perform structured prompt sentence generation that systematically encodes: (i) the current condition information, (ii) the user's past menu selection history, and (iii) explicit constraints on output format and resource usage. Consequently, the generative AI model often returns outputs that are inconsistent in structure or insufficiently tailored, which forces additional parsing, post-processing, and repeated requests on the server side. This increases processor load, network traffic, and memory operations, degrading overall system efficiency.

Third, known systems do not tightly integrate inventory ingredient information and use-by-date information into the prompt construction workflow at the processor level. Many systems merely display lists of ingredients or highlight expiring items, leaving the generative AI model unaware of concrete constraints on inventory usage or food waste reduction. This separation results in a suboptimal computational pipeline in which the processor must reconcile generated menus with inventory constraints after the fact, requiring additional corrective computation and database updates.

Fourth, from a system-architecture perspective, existing solutions do not define a consistent, machine-oriented format for converting the generative AI output into structured data that can be persistently stored and reused to generate subsequent prompt sentences. Without such a closed loop between generated menu information, user selection events, and future prompt generation, the processor cannot incrementally refine its behavior based on accumulated history. This limits the ability of the system to adaptively optimize processing paths and data flows as user data grows.

Accordingly, there is a need for an improved computer-implemented system and server-side processing architecture in which a processor: (1) acquires user condition information through a controlled interface; (2) reads and analyzes past menu selection history in storage; (3) generates a structured, constraint-rich prompt sentence for a generative AI model; (4) efficiently obtains and structurally converts menu information generated by the model; and (5) appends such information and user selection results back into storage for use in subsequent prompt generation. By technically coupling these operations, the system can improve the efficiency, consistency, and adaptability of the overall computational pipeline, reduce redundant computation and network usage, and provide more predictable and structured outputs from the generative AI model, thereby improving the functioning of the computer system itself.

The specific processing by the specific processing unit 290 of the data processing device 12 in Example 1 is realized by the following means.

The present invention provides a server comprising a processor and a storage device, the processor being configured to control an information input and output interface to acquire condition information regarding a cooking target from a user; read, from the storage device, past menu selection history information associated with the user and acquire the past menu selection history information together with the condition information; analyze the condition information and the menu selection history information to generate a prompt sentence that instructs a generative artificial intelligence model to generate a menu and that defines an output format and constraint conditions; transmit the prompt sentence to an external generative artificial intelligence model and acquire menu information generated by the generative artificial intelligence model based on the prompt sentence; convert the acquired menu information into a predetermined data structure and transmit the converted menu information to a terminal device of the user so as to be presented via the information input and output interface; and append and store the acquired menu information and a menu selection result by the user as the menu selection history information in the storage device, and reflect the menu selection history information in generation of a subsequent prompt sentence. This enables the server to implement an improved, closed-loop computational pipeline in which user condition information, historical selection data, and inventory constraints are systematically encoded into structured prompt sentences for the generative AI model, thereby producing more consistent and machine-parseable outputs, reducing redundant processing and network interactions, and enhancing the overall efficiency and adaptability of the computer-implemented menu generation system.

The term “processor” refers to an electronic data processing unit, such as a central processing unit or other computing circuitry, that executes instructions to perform arithmetic, logical, control, and input/output operations.

The term “storage device” refers to a hardware component or combination of components, such as a memory device or non-volatile storage medium, that stores data and program instructions used by the processor.

The term “information input and output interface” refers to a hardware and/or software interface, such as a graphical user interface, application programming interface, or communication interface, through which the system acquires information from a user and presents information to the user.

The term “condition information” refers to information indicating one or more requirements, constraints, or preferences specified by a user regarding a cooking target, including at least one of a health-related condition, a nutrition-related condition, a cooking time condition, or an inventory-related condition.

The term “cooking target” refers to a meal, dish, or set of dishes for which a menu or recipe is to be generated.

The term “menu selection history information” refers to data indicating past menus or recipes that have been generated, presented, and/or selected by a user, including at least one of identifiers, timestamps, ingredient characteristics, or selection results associated with such menus.

The term “preference tendency information” refers to information derived from menu selection history information that represents patterns or tendencies in a user's past selections, including at least one of frequently selected ingredient types, avoided ingredient types, preferred cooking times, or favored dish categories.

The term “inventory ingredient information” refers to information indicating ingredients that are available for use, including at least one of ingredient types, quantities, storage locations, or acquisition dates.

The term “use-by-date information” refers to information indicating an expiration date, best-before date, or recommended usage limit associated with an inventory ingredient.

The term “prompt sentence” refers to text data in natural language that is generated by the processor and supplied as input to a generative artificial intelligence model, the text data including instructions, conditions, and format specifications for menu generation.

The term “generative artificial intelligence model” refers to a machine learning model configured to generate text or other content in response to input data, the model being trained on large-scale data and capable of producing menu information based on a prompt sentence.

The term “menu information” refers to information representing one or more proposed menus or recipes, including at least one of dish titles, estimated cooking times, ingredient lists, quantities, and cooking steps generated by a generative artificial intelligence model.

The term “predetermined data structure” refers to a structured representation format, such as a data schema or data model, defined in advance for storing and processing menu information, including at least one of a hierarchical structure, a list structure, or a key-value structure.

The term “terminal device” refers to an electronic device operated by a user, such as a mobile communication device, a portable computing device, or a stationary computing device, that communicates with the server to send condition information and receive menu information.

The term “constraint conditions” refers to conditions that restrict or guide the generation of menu information by a generative artificial intelligence model, including at least one of formatting constraints, dietary constraints, cooking time constraints, cost constraints, or resource usage constraints.

The term “health condition” refers to condition information relating to health aspects of cooking, including at least one of calorie restrictions, nutrient balance, dietary restrictions, or medical considerations.

The term “nutrition condition” refers to condition information specifying nutritional requirements or goals, including at least one of desired macronutrient ratios, micronutrient targets, or avoidance of certain nutrients.

The term “cooking time condition” refers to condition information specifying allowable or desired time for preparing and cooking a dish or menu.

The term “food waste reduction” refers to a reduction in the quantity of edible ingredients discarded, achieved by generating menus that preferentially use ingredients approaching their use-by dates or ingredients that are otherwise likely to be wasted.

The term “resource consumption reduction” refers to a reduction in consumption of resources, including at least one of food ingredients, energy, or water, achieved through menu generation that considers efficiency and usage constraints.

The term “economic benefit” refers to a benefit to the user resulting from decreased waste or improved efficiency, including at least one of reduced expense for ingredients, reduced utility costs, or improved utilization of purchased items.

The term “closed-loop computational pipeline” refers to a sequence of processing operations in which output data, including menu information and user selection results, is fed back into storage and subsequently used as input or context for future prompt sentence generation and menu generation.

The term “external generative artificial intelligence model” refers to a generative artificial intelligence model executed on a computing resource outside the server, such as a remote computing service, and accessed by the server through a communication network.

In the following embodiments, a server, a terminal, and a user cooperate to implement a system that generates and presents menus using a generative AI model based on prompt sentences. The embodiments are described in sufficient detail for a person skilled in the art to make and use the invention, and various modifications and alternative configurations are also contemplated.
1. Hardware and software configuration of server and terminal
Server:

Server is implemented by one or more computing devices that collectively function as a network-accessible server system. Server includes at least one processor, a main memory, a non-volatile storage device, and a network interface. The processor may be a central processing unit, a graphics processing unit, or another programmable processing circuit. The main memory may be a volatile memory device such as dynamic random access memory. The storage device may be a non-volatile memory such as a solid-state drive or a magnetic disk. The network interface may include an Ethernet adapter, a wireless communication module, or another communication interface connected to a communication network such as the Internet.

Server executes an operating system, such as a general-purpose server operating system, and an application stack including a web server component (for example, server may execute an HTTP server implemented in an industry-standard web server framework), an application framework (for example, a web application framework for handling HTTP requests, routing, and JSON parsing), and one or more software modules implementing prompt generation, generative AI model communication, menu post-processing, and history management. Server is connected to at least one storage device that stores user data, including menu selection history information, inventory ingredient information, and configuration parameters for prompt sentence generation. Server may employ a relational database management system or a non-relational database system, together with an object-relational mapping layer or database driver.
Terminal:

Terminal is implemented by a user-operated computing device, such as a smartphone, a tablet, a laptop computer, or a desktop computer. Terminal includes a processor, a memory, a display, an input device such as a touchscreen or keyboard, and a network interface such as a wireless transceiver. Terminal executes an operating system, such as a mobile operating system or a desktop operating system, and a client application that communicates with server via a network using a communication protocol such as HTTPS.

Terminal executes a user interface layer implemented using a UI framework or browser-based technologies. Terminal provides an information input and output interface that allows user to input condition information and to view generated menu information. Terminal may use local storage or an internal database to temporarily store user inputs or cached menu information, but the main processing related to prompt generation and generative AI interaction is executed by server.
Generative AI environment:

Server communicates with an external generative AI model via a network. The generative AI model is implemented by a neural network model that executes on external computing infrastructure, such as a cloud-based cluster of processing units with specialized hardware for matrix computations. The generative AI model uses a transformer-based architecture including multiple encoder-decoder or decoder-only layers, multi-head self-attention mechanisms, and feed-forward sub-networks. The model parameters are stored as numerical weight matrices and bias vectors. The generative AI model receives tokenized text input corresponding to a prompt sentence and outputs tokenized text which is converted back into natural language.
2. Data structures used by the server

Server uses concrete data structures for efficient and reproducible processing of condition information, menu selection history information, and generated menu information.

Server stores condition information as a structured record in memory, including at least the following fields:
a field for a health-related condition (for example, a string indicating “healthy,” “low-salt,” or “low-fat”);
a field for a nutrition-related condition (for example, an encoded representation of macronutrient or micronutrient preferences);
a field for a cooking time condition (for example, an integer representing maximum cooking time in minutes);
a field for a target group (for example, a string indicating “for kids,” “for adults,” or “for seniors”);
a field for inventory ingredient information (for example, a list of ingredient identifiers, quantities, and use-by dates).

Server stores menu selection history information in the storage device as records including at least:
a user identifier;
a menu identifier or title;
ingredient and attribute tags for the menu (for example, tags indicating “chicken,” “fish,” “non-dairy,” “vegetarian”);
a timestamp of the generation and a timestamp of the selection;
flags or scores indicating user satisfaction, if available.

Server uses a structured menu information representation when storing and transmitting menus. A record of a generated menu includes:
a title field;
an estimated cooking time field;
a list of ingredient entries, each with a name, quantity, and optional unit;
a list of cooking steps, ordered and expressed as text strings;
optional nutritional metadata, if generated.

By using explicit data structures rather than unstructured text only, server reduces ambiguity and minimizes parsing overhead in subsequent processing, thereby improving computational efficiency and reducing memory fragmentation.
3. Prompt sentence generation and model-side processing

Server converts the condition information and the menu selection history information into a prompt sentence that guides the generative AI model. Server performs the following technical operations to generate a prompt sentence in a structured manner.

Server retrieves, from the storage device, menu selection history information associated with the user. Server executes a preference extraction module that aggregates ingredient frequencies, identifies ingredients that are rarely or never selected, computes preferred cooking time ranges, and extracts patterns relating to health and nutrition. This module may compute numerical statistics such as counts and normalized frequencies of tags, and may determine threshold-based preference categories (for example, ingredients selected above a frequency threshold are treated as preferred, and ingredients below another threshold are treated as avoided).

Server then uses a prompt template engine to assemble a prompt sentence that encodes:
the current condition information;
the extracted preference tendency information;
explicit constraints on output structure;
constraints on inventory use and food waste reduction.

For example, server may generate the following prompt sentence:
“You are an expert home cook and nutritionist. The user wants healthy, kid-friendly dinner menus that can be cooked in 15 minutes or less. The user frequently chooses chicken-based dishes and prefers low-sodium and non-dairy meals. Please propose four menu ideas that reflect these preferences. For each idea, provide: (1) a title, (2) an estimated cooking time, (3) a list of ingredients with approximate amounts, and (4) concise, step-by-step cooking instructions, keeping salt usage low and avoiding dairy products.”

Server may generate alternative prompt sentences depending on different condition information. For example, when inventory ingredient information and use-by dates are important, server may generate:
“The user wants to reduce food waste and energy consumption. Consider the following inventory ingredients that should be used soon: chicken breast (use-by date tomorrow), broccoli (use-by date tomorrow), and tomatoes (use-by date in two days). Please propose three healthy dinner menus that can be cooked in 20 minutes or less using these ingredients as primary components. For each menu, provide a title, estimated cooking time, ingredients with approximate amounts, and step-by-step instructions. Prioritize using ingredients with the nearest use-by dates and avoid introducing many additional ingredients.”

By explicitly encoding such constraints and structure into prompt sentences, server causes the generative AI model to produce outputs that are already formatted in a consistent manner and aligned with inventory conditions. This reduces downstream parsing and correction operations, which in turn improves processing speed and reduces memory usage on server.
4. Internal structure and learning behavior of the generative AI model

The generative AI model used by server employs a transformer architecture that includes an embedding layer, multiple stacked transformer blocks, and an output projection layer. Each transformer block includes a multi-head self-attention sub-layer and a position-wise feed-forward sub-layer. The model processes input text as a sequence of tokens, where each token is represented as a vector. The self-attention mechanism computes attention scores between all token positions, enabling the model to capture long-range dependencies in natural language input.

The generative AI model is trained using large-scale text data that includes recipes, menus, and general-purpose natural language sentences. During training, the model receives input sequences and is trained to predict the next token in the sequence using a loss function such as cross-entropy loss. The model updates its weight parameters by gradient-based optimization methods, for example, stochastic gradient descent with adaptive learning rates.

The training process may include techniques such as mini-batch training, dropout regularization, and learning rate scheduling, to improve generalization performance and prevent overfitting.

In some implementations, the generative AI model may be fine-tuned using domain-specific data such as structured recipe datasets and user interaction logs. Fine-tuning may adjust parameters so that the model places higher importance on constraints relevant to menu generation, such as ingredient lists and procedural steps. This adaptation improves the accuracy and reliability of the generated menu information.

By employing a transformer-based generative AI model with explicit, constraint-rich prompt sentences, server leverages complex multi-head self-attention and learned weight matrices to resolve conflicting constraints (for example, healthy, kid-friendly, and low-sodium, while using expiring ingredients) that are difficult to handle with rule-based approaches. The coupling of the preference statistics, inventory constraints, and structured output specification within the prompt sentence leads to a technical improvement in the generative processing pipeline, enabling more reliable and efficient text generation compared to unstructured or naive prompts.
5. Technical effects and improvement of computer technology

Server improves computer technology by modifying the way data is stored, processed, and transmitted in connection with the generative AI model. Specifically, server creates a closed-loop computational pipeline where output menu information and user selection results are systematically appended into storage and fed back into subsequent prompt sentence generation.

Because server encodes past selection history and inventory constraints into the prompt sentences, the generative AI model produces menus that require less post-processing and fewer corrective requests. This reduces the number of network round trips between server and the external generative AI infrastructure, thereby reducing communication load. Furthermore, by defining a predetermined data structure for menu information at the server side, server can parse model outputs using deterministic parsing rules, reducing CPU time and avoiding complex error handling that would be necessary for inconsistent outputs.

In contrast to simply automating human selection of recipes, server implements a non-conventional prompt construction and data-management strategy that is specifically tailored to the architecture of a transformer-based generative AI model. The strategy optimizes how the model's input space is conditioned on structured user and inventory data. This arrangement yields improved prediction accuracy and reduces the overall number of tokens that must be processed, because the prompt sentence can directly instruct the model to output only the necessary menu components, thus saving computation and memory in the external AI system.

Additionally, the inclusion of use-by-date information and inventory ingredient information into the prompt sentences allows server to offload part of the combinatorial search for ingredient combinations to the generative AI model while ensuring that the combinations are constrained. Because the model receives explicit instructions about which ingredients must be prioritized, server can avoid running separate optimization or constraint-satisfaction algorithms on its own hardware, thereby reducing local CPU load.

The closed-loop update of menu selection history also leads to a more compact representation of user preferences over time. Server can periodically compress historical logs into aggregated preference vectors, reducing storage volume and speeding up subsequent history retrieval. This contributes to improved data management, including faster query execution and lower storage overhead.
6. Distinction from conventional human or rule-based processing

User may specify conditions such as “healthy,” “for kids,” “15 minutes,” or “low-salt,” but user does not manually search and combine recipes. Likewise, server does not rely solely on fixed rule-based matching of tags to a static recipe database. Instead, server converts high-level user conditions and statistical preference patterns into prompt sentences that are specifically structured to leverage the generative AI model's ability to synthesize new menu combinations.

For example, when user repeatedly selects chicken-based menus, server does not merely increase a score for chicken recipes in a predefined list. Instead, server explicitly mentions in the prompt sentence that “The user frequently chooses chicken-based dishes and prefers low-sodium and non-dairy meals,” causing the generative AI model to adjust its internal attention patterns and token predictions to favor such combinations. This behavior is distinct from conventional content-based filtering and represents a technical integration of statistical preference modeling with neural text generation.

Moreover, server defines non-standard, machine-oriented rules for output structure in the prompt sentences. Server's prompt sentences require the generative AI model to output exactly defined components such as “(1) a title, (2) an estimated cooking time, (3) a list of ingredients with approximate amounts, and (4) concise, step-by-step cooking instructions.” This reduces ambiguity and allows server to apply predictable parsing routines to the output. The use of such structured prompts and consistent output schemas is a non-conventional technique for controlling generative text models in a way that specifically improves downstream computational efficiency.
7. Alternative embodiments and variations

Server may implement different strategies for summarizing menu selection history. In one embodiment, server computes a vector representation of each menu based on ingredient tags and health attributes and then aggregates these vectors over the user's history to produce a dense preference vector. Server may then convert the preference vector into textual description in the prompt sentence (for example, “The user often chooses high-protein, low-carbohydrate dishes with poultry and vegetables.”). In another embodiment, server may categorize users into preference clusters and include cluster-specific constraints in the prompt.

Server may vary the degree of structure in the prompt sentence. In one embodiment, prompt sentences include explicit delimiter tokens or headings such as “Title:”, “Time:”, “Ingredients:”, and “Steps:”. The use of such consistent delimiters simplifies parsing and reduces error rate when converting generated text into the predetermined data structure. In another embodiment, server uses numbered steps and numbered lists of ingredients to facilitate deterministic parsing.

Server may also change the way inventory information is integrated. In one embodiment, server lists inventory ingredients and their use-by dates in the prompt sentence. In another embodiment, server computes a list of “urgent” ingredients that should be used soon and explicitly orders the generative AI model to place those ingredients in the first menu proposals. For example, server may use a prompt sentence such as:
“Among the inventory ingredients, chicken breast and broccoli must be used today. Please ensure that at least two of the proposed menus use chicken breast and broccoli as main ingredients. Clearly indicate in each menu which inventory ingredients are used.”

Terminal may be implemented in various forms, such as a native mobile application, a web application, or a hybrid application. Regardless of specific implementation, terminal operates as a client that collects condition information, sends it to server, and displays the resulting menu information based on the data structure received from server. Terminal may also assist in reducing network usage by caching recent menu information and reusing it when similar conditions are entered.
8. Example usage scenario

User launches a client application on terminal and inputs condition information. User may enter text such as “healthy, kid-friendly, 15 minutes,” and may specify that only ingredients currently present in the refrigerator should be used. Terminal sends this condition information to server.

Server retrieves selection history, identifies that user often selects chicken-based, non-dairy recipes, and detects that chicken breast and broccoli are nearing their use-by dates. Server generates a prompt sentence such as:
“You are an expert home cook and nutritionist. The user wants healthy, kid-friendly dinners that can be cooked in 15 minutes or less. The user frequently chooses chicken-based, non-dairy meals. The following inventory ingredients should be used soon: chicken breast (use-by date today) and broccoli (use-by date today). Please propose four menu ideas that use these ingredients as main components. For each idea, provide a title, an estimated cooking time, a list of ingredients with approximate amounts, and concise, step-by-step cooking instructions. Avoid dairy products and keep salt usage low.”

Server transmits this prompt sentence to the external generative AI model. The generative AI model processes the prompt sentence using its transformer layers, computing attention scores between tokens and generating an output sequence that contains four menus, each described with a title, time, ingredients, and steps. Server receives this output, parses titles, times, ingredients, and steps according to the expected structure, and converts the parsed content into the predetermined data structure. Server then stores the resulting menu information and transmits it to terminal.

Terminal presents the menus to user in a structured view. User selects one menu to cook.

Terminal notifies server of the selection, and server updates the menu selection history accordingly. In subsequent use, server uses the updated history to generate more refined prompt sentences and menu proposals.

Through these embodiments, server, terminal, and the generative AI model cooperate in a technically integrated manner. The system achieves improved computational efficiency, reduced network load, better-structured data management, and more accurate adherence to user and inventory constraints compared with conventional systems that either rely solely on static recipe databases or use generative AI models without structured prompt sentence generation and closed-loop history integration.

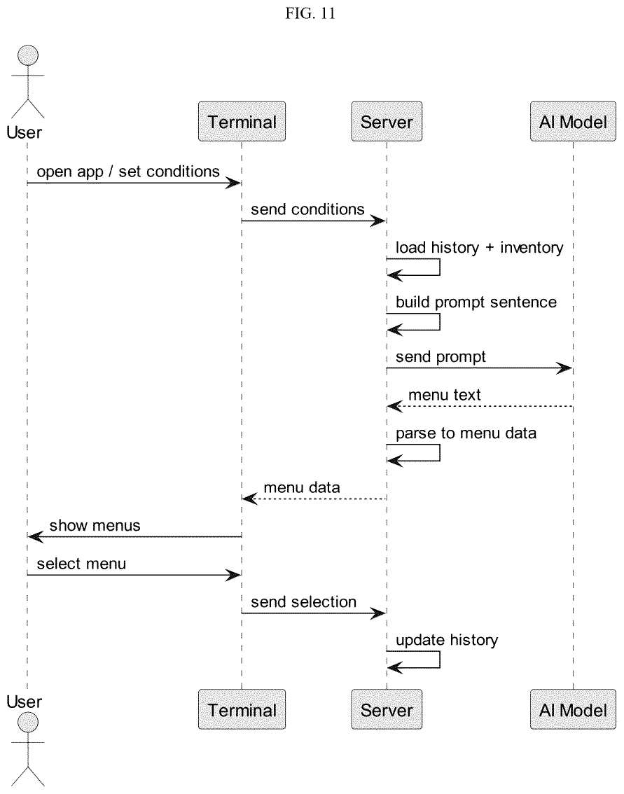

The following describes the processing flow using FIG. 11.

### Step 1

User starts a client application on the terminal and prepares condition input.
User launches a menu-generation application on the terminal and waits for the main screen to appear.
Terminal loads the user interface layout, allocates memory for input fields (for example, text boxes, check boxes, drop-down lists), and initializes an internal condition object with empty or default values.
Input: none (application initialization).
Output: an initialized condition object in terminal memory, for example with fields for health condition, nutrition condition, cooking time condition, target group, and inventory-related options.

### Step 2

User inputs condition information via the terminal interface.
User types text such as “healthy,” “for kids,” and “15 minutes,” and optionally selects options such as “low-salt,” “non-dairy,” or “use ingredients in refrigerator.”
Terminal captures key events and touch/click events, updates the internal condition object, and displays the current selections on the screen.
Terminal performs basic validation, such as confirming that the time field is a positive integer and that required fields are not empty.
Input: user interactions (keystrokes, touches, clicks).
Output: a populated condition object in terminal memory containing values for health-related conditions, nutrition-related conditions, cooking time condition, target group, and inventory-related flags.

### Step 3

Terminal packages and transmits the condition information to server.
Terminal converts the internal condition object into a standardized representation (for example, a JSON message) and prepares a network request to server using a secure communication protocol.
Terminal attaches a user identifier or session token, if available, and sends the request over a network interface.
Input: the populated condition object and optional user identifier.
Output: a network message transmitted to server that encodes the condition information and identification data.

### Step 4

Server receives and parses the condition information from terminal.
Server accepts the incoming network message through a web server component, passes the message to an application layer, and parses the structured data (for example, parses JSON into an internal data structure).
Server validates the received condition information, checking types and required fields, and logs the request for monitoring or debugging.
Input: the network message containing condition information and user identifier.
Output: a server-side condition record stored in server memory, which includes fields aligned with the claim terminology (health condition, nutrition condition, cooking time condition, inventory ingredient flags, and user identifier).

### Step 5

Server retrieves menu selection history information associated with the user.
Server uses the user identifier obtained from the condition record to query a storage device via a database interface.
Server executes a query that retrieves past menu selection history records, including menu identifiers, ingredient tags, timestamps, and selection flags.
Server loads these records into memory and may limit them to a recent time window to reduce processing cost.
Input: the server-side condition record including the user identifier.
Output: a collection of menu selection history records associated with the user, stored in server memory.

### Step 6

Server analyzes the menu selection history information to derive preference tendency information.
Server executes a preference analysis module that counts the frequency of ingredient tags, dish categories, and health attributes across the retrieved history records.
Server computes normalized frequencies and identifies preferred and avoided ingredients based on thresholds.
Server may calculate distributions of cooking times in the history and infer preferred ranges.
Input: the collection of menu selection history records.
Output: a preference profile in server memory, including lists of preferred ingredient types, avoided ingredient types, typical cooking time ranges, and favored dish attributes.

### Step 7

Server retrieves inventory ingredient information and use-by-date information when available.
Server checks whether the condition record indicates that inventory data should be used.
Server queries a storage device or an external appliance interface to obtain records indicating current ingredients, quantities, and use-by dates.
Server filters these records to identify ingredients that are close to their use-by dates and classifies them as urgent ingredients.
Input: the server-side condition record and inventory database entries or appliance data.
Output: an inventory summary, including a list of available ingredients and a subset of urgent ingredients that should be consumed soon.

### Step 8

Server composes a structured prompt sentence for the generative AI model.
Server invokes a prompt generation routine that takes the condition record, the preference profile, and the inventory summary as inputs.
Server applies a template-based or rule-based algorithm that inserts values from these data structures into a natural-language prompt pattern.
Server encodes explicit instructions about the desired output structure, such as specifying that each menu must include a title, an estimated cooking time, a list of ingredients with approximate amounts, and step-by-step cooking instructions.
For example, server may construct the following prompt sentence:
“You are an expert home cook and nutritionist. The user wants healthy, kid-friendly dinner menus that can be cooked in 15 minutes or less. The user frequently chooses chicken-based, non-dairy meals. The following inventory ingredients should be used soon: chicken breast (use-by date today) and broccoli (use-by date today). Please propose four menu ideas that use these ingredients as main components. For each idea, provide a title, an estimated cooking time, a list of ingredients with approximate amounts, and concise, step-by-step cooking instructions. Avoid dairy products and keep salt usage low.”
Input: the condition record, the preference profile, and the inventory summary.
Output: a single prompt sentence in natural language, stored as a text string in server memory.

### Step 9

Server transmits the prompt sentence to the external generative AI model.
Server formats a request to the generative AI environment, placing the prompt sentence into a designated input field, and sets model parameters such as maximum output length and creativity level.
Server sends the request through a network interface, using a defined API protocol for the generative AI service.
Input: the prompt sentence and configuration parameters for the generative AI model.
Output: a model request message transmitted to the external generative AI infrastructure.

### Step 10

Server receives text output from the generative AI model.
Server waits for a response from the generative AI service.
The generative AI model processes the prompt sentence by tokenizing the text, applying multiple transformer layers with self-attention and feed-forward operations, and generating an output sequence of tokens, which is decoded to natural-language text describing multiple menus.
Server receives the resulting text via the API and stores the text in memory as raw model output.
Input: the model's response message containing generated text.
Output: raw output text in server memory, typically representing multiple menu proposals in natural language.

### Step 11

Server parses and converts the generated text into a predetermined data structure.
Server runs a parsing routine that recognizes structural elements in the output text, such as menu titles, cooking times, ingredient lists, and procedure steps, based on delimiters, headings, or numbering enforced by the prompt sentence.
Server splits the text into individual menu entries, extracts each component, and validates data types (for example, ensuring that cooking times are numeric).
Server constructs a structured menu representation, assigning each menu an identifier and organizing its components into fields corresponding to the predetermined data structure.
Input: raw output text from the generative AI model.
Output: a structured set of menu information objects containing titles, cooking times, ingredients, and step sequences.

### Step 12

Server stores generated menu information and updates menu selection history.
Server writes the structured menu information to the storage device, associating each menu with the user identifier and a generation timestamp.
Server may also pre-compute and store ingredient tags and health attributes for each generated menu to speed up future preference analysis.
Input: the structured set of menu information objects and the user identifier.
Output: updated records in the storage device including new generated-menu entries linked to the user.

### Step 13

Server prepares a response to terminal containing the structured menu information.
Server selects a subset or all of the generated menus, packages them into a response message, and includes additional metadata such as the generation time and optional explanations.
Server serializes the menu objects into a format suitable for transmission, such as a structured representation, and sends the response back to terminal over the network.
Input: the structured menu information objects intended for presentation.
Output: a network response message transmitted to terminal with structured menu data.

### Step 14

Terminal receives the structured menu information and renders it for user.
Terminal parses the response message, reconstructs the menu items into internal UI data models, and prepares display elements such as cards or list items for each menu.
Terminal formats ingredient lists and step-by-step instructions into readable layouts and may highlight menus that match strong preference patterns or urgent inventory usage.
Input: the network response message containing structured menu data.
Output: a rendered set of visual elements on the terminal display, showing menu titles, times, ingredients, and steps.

### Step 15

User reviews the proposed menus and selects a preferred menu.
User scrolls through the displayed menus on terminal, compares cooking times and ingredients, and chooses one menu for actual cooking by tapping or clicking a selection control.
Terminal captures this selection event and records which menu has been chosen.
Input: the displayed menu options and user's selection action.
Output: a selection record in terminal memory identifying the chosen menu.

### Step 16

Terminal reports the user's menu selection back to server.
Terminal constructs a selection notification message that includes the user identifier, the selected menu identifier or title, and the selection timestamp.
Terminal sends this message to server via the same secure communication protocol.
Input: the local selection record and user identifier.
Output: a network message transmitted to server describing the specific menu selected by user.

### Step 17

Server records the selection event and updates preference-related statistics.
Server receives the selection message, parses it, and inserts a corresponding selection event into the menu selection history records in the storage device.
Server updates counters and aggregated statistics related to ingredient usage, dietary attributes, and cooking times for the user, adjusting the preference profile to reflect the new choice.
Input: the selection event message containing user identifier and selected menu information.
Output: updated menu selection history and updated preference statistics in storage, ready to be used as input to future prompt sentence generation.

### Application Example 1

Description follows regarding a flow of the specific processing in an Application Example 1. The units of the system described below are implemented by the data processing device 12 and the smart device 14. The data processing device 12 is called a “server” and the smart device 14 is called a “terminal”.

Conventional computer-implemented menu recommendation systems typically rely on static rule sets or simple filtering logic that operate on user-entered conditions and stored recipe databases. Such systems suffer from several technical limitations.

First, conventional systems are not designed to dynamically construct and supply an optimized prompt sentence to a generative AI model. As a result, when a generative AI model is used, input data such as user conditions, ingredient availability, health constraints, and delivery constraints are passed in an unstructured or suboptimal manner. This leads to inefficient utilization of the generative AI model's computational capabilities, causing redundant processing, increased latency, and inconsistent quality of generated menu information.

Second, traditional architectures do not tightly integrate sensor-acquired ingredient information from a storage apparatus with downstream data processing and order execution. Ingredient data obtained from detection devices (e.g., sensors in a storage apparatus) is often processed independently of menu generation logic. This separation prevents the processor from performing low-level data operations—such as structured comparison between required ingredient information and available ingredient information—in a unified pipeline. As a consequence, shortage ingredient information is derived using ad hoc or manual procedures, which increases computational overhead, causes data inconsistency across components, and degrades system responsiveness.

Third, existing systems are inadequate in optimizing multiple technical objectives simultaneously, such as reducing food waste by considering expiration date information and suppressing power consumption by coordinating menu decisions with operation information of a storage apparatus. In conventional systems, these aspects are handled, if at all, by loosely coupled subsystems or by post-processing steps outside the core processing pipeline. This fragmented architecture prevents the processor from executing coordinated data processing operations that jointly evaluate ingredient usage, expiration dates, and device operation parameters. Hence, the overall system cannot efficiently compute order information that balances menu suitability with resource utilization, causing suboptimal performance in terms of storage apparatus operation and data processing throughput.

Fourth, current implementations lack a well-defined end-to-end computational flow that begins with user condition input, proceeds through prompt sentence generation and generative AI inference, and continues to automatic order creation and transmission to a delivery service device. The absence of such an integrated flow forces developers to implement multiple independent interfaces and transformations, leading to duplicated logic, increased latency due to repeated data conversions, and a higher risk of processing errors. This results in a system that is less scalable, harder to maintain, and less efficient in using network, processor, and memory resources.

Accordingly, there is a need for an improved computer-implemented system and associated processing techniques in which a processor is configured to (i) acquire user condition information and sensor-based ingredient information, (ii) generate a structured prompt sentence for a generative AI model in a manner optimized for subsequent machine processing, (iii) analyze the generated menu information to compute shortage ingredient information, and (iv) automatically generate order information that reduces food waste and suppresses power consumption. Such a system should improve the overall technical performance of the menu recommendation and ordering pipeline, including reduced processing latency, improved consistency of data transformations, and more efficient usage of computing and storage resources.

The specific processing by the specific processing unit 290 of the data processing device 12 in Application Example 1 is realized by the following means.

The present invention provides a server comprising a processor and at least one memory storing instructions that, when executed by the processor, cause the processor to receive condition information regarding a menu from a user via an information input/output unit, acquire ingredient information in a storage apparatus by using a detection device, generate a prompt sentence including the condition information and reflecting the ingredient information based on the acquired ingredient information and the condition information, and input the prompt sentence to a generative information processing model to cause the generative information processing model to execute a generation process of menu information; analyze the menu information output from the generative information processing model to extract required ingredient information for each menu; compare the required ingredient information with the ingredient information in the storage apparatus to calculate shortage ingredient information; present, to the user, the menu information including the shortage ingredient information and acquire menu selection information and order confirmation information from the user; and store order information in a storage device based on the order confirmation information and transmit the order information to a delivery service device, wherein the processor is further configured to structure health state information, subject attribute information, service time constraint information, and ingredient information in the storage apparatus included in the condition information and incorporate the structured information into the prompt sentence such that the generative information processing model generates menu information that conforms to the health state information and satisfies the service time constraint information, and wherein the processor is still further configured to generate the order information such that economic benefit is provided to the user by selecting the required ingredient information so as to reduce food waste amount based on the required ingredient information, the ingredient information in the storage apparatus, and expiration date information, and by determining the menu information so as to suppress power consumption amount based on operation information of the storage apparatus and the menu information. This enables an integrated, computer-implemented processing pipeline in which the construction of the prompt sentence, the execution of generative AI inference, the calculation of shortage ingredient information, and the generation of order information are coordinated at the processor level, thereby improving utilization efficiency of the generative AI model, reducing overall processing latency, ensuring consistent data transformations across components, and optimizing resource usage in both the storage apparatus and the server-side computing environment.

The term “processor” refers to a hardware computation unit, or a combination of hardware computation units, that executes machine-readable instructions to perform data processing operations, including receiving data, generating a prompt sentence, invoking a generative information processing model, analyzing menu information, calculating shortage ingredient information, and generating order information.

The term “memory” refers to a non-transitory computer-readable medium that stores instructions and data to be used by the processor, including instructions for generating the prompt sentence, analyzing menu information, and managing order information.

The term “information input/output unit” refers to a hardware and software interface configured to exchange information between the server and an external device, such as a user terminal, including receiving condition information from a user and presenting menu information to the user.

The term “detection device” refers to a sensing component or a group of sensing components associated with a storage apparatus, configured to detect physical states related to ingredients, such as presence, quantity, or identification of items, and to output corresponding ingredient information in an electronic format.

The term “storage apparatus” refers to an equipment for storing perishable or non-perishable items, including but not limited to refrigeration equipment and freezing equipment, which is capable of housing ingredients and providing operational information such as temperature or power state.

The term “ingredient information” refers to data representing at least one attribute of an item stored in the storage apparatus, including type, quantity, unit, and optionally expiration date information.

The term “condition information” refers to data representing user-specific requirements for a menu, including but not limited to health state information, subject attribute information, service time constraint information, taste preference information, and budget-related information.

The term “prompt sentence” refers to a structured text sequence generated by the processor, which includes at least the condition information and the ingredient information, and which is supplied as input to a generative information processing model to cause the model to generate menu information.

The term “generative information processing model” refers to a machine learning model, such as a neural network-based generative AI model, that processes the prompt sentence and outputs generated information including menu information in a natural language or structured format.

The term “menu information” refers to data representing at least one proposed menu, including a menu title, a description, required ingredient information, and optionally preparation or service-related metadata.

The term “menu analysis unit” refers to a functional component implemented by the processor and memory, configured to parse and analyze menu information output from the generative information processing model and to extract required ingredient information for each menu.

The term “required ingredient information” refers to data within the menu information that specifies which ingredients and what quantities are needed to realize a particular menu.

The term “shortage ingredient information” refers to data representing those ingredients, or corresponding quantities, that are required according to the required ingredient information but are not sufficiently available according to the ingredient information of the storage apparatus.

The term “shortage information calculation unit” refers to a functional component implemented by the processor and memory, configured to compare the required ingredient information with the ingredient information in the storage apparatus and to calculate shortage ingredient information.

The term “order reception unit” refers to a functional component implemented by the processor and memory, configured to present menu information to the user, receive menu selection information and order confirmation information from the user, and output corresponding order information.

The term “order information” refers to data representing a confirmed order, including at least a selected menu identifier, required ingredients, user identification information, delivery destination information, and service time information.

The term “storage device” refers to a non-transitory storage medium or storage subsystem, such as a database system, configured to store order information, ingredient information, and associated management data for subsequent retrieval and processing.

The term “delivery service device” refers to a computing apparatus or service endpoint associated with a delivery service provider, which receives order information from the server and manages logistics operations such as assignment of delivery personnel and scheduling of deliveries.

The term “health state information” refers to data within the condition information that indicates a health-related requirement or constraint of a user or subject, such as dietary restrictions, nutritional preferences, or medical conditions.

The term “subject attribute information” refers to data within the condition information that represents characteristics of a person or group for whom the menu is intended, such as age group, number of persons, or specific lifestyle attributes.

The term “service time constraint information” refers to data within the condition information that specifies a temporal limitation for providing the menu, such as a maximum preparation time, a maximum delivery time, or a desired serving time window.

The term “expiration date information” refers to data associated with an ingredient that indicates a recommended or mandatory use-by date or best-before date, which is used in determining food waste reduction.

The term “operation information of the storage apparatus” refers to data representing operational parameters of the storage apparatus, such as internal temperature, compressor state, power consumption, operating mode, or door open/close history, which can be used to estimate or control power consumption.

The term “economic benefit” refers to an advantage provided to the user through reduced overall cost or resource usage, including reductions in food waste due to consideration of expiration date information and reductions in power consumption due to consideration of operation information of the storage apparatus.

In one embodiment, a server cooperates with a terminal and a storage apparatus to implement the claimed system. The server includes at least one processor and at least one memory that stores program instructions and data structures. The terminal includes a user interface subsystem implemented, for example, by a touch display and a mobile operating system. The storage apparatus includes a refrigeration unit equipped with one or more sensors functioning as a detection device.

The server executes an operating system such as a general-purpose server operating system and hosts an application stack including a web server component and an application framework. The server further maintains a database system, for example a relational database management system, as a storage device to store ingredient information, menu information, and order information. The server communicates with an external generative AI platform that provides a generative AI model, which is an instance of a neural network-based generative information processing model.

The terminal executes an application, such as a native application on a mobile platform or a web application rendered in a browser. The terminal provides an information input/output unit to the user. The terminal displays input controls and menu options and sends user-generated data to the server via a communication network using a network protocol such as HTTPS. The user operates the terminal to enter condition information including health state information, subject attribute information, and service time constraint information. The terminal transmits this condition information as structured data to the server.

The storage apparatus includes a detection device consisting of multiple sensor elements, such as weight sensors, optical imaging sensors, and radio-frequency identification sensors.

The storage apparatus acquires ingredient information by detecting an item stored in an internal compartment, mapping sensor signals to item identifiers and quantities, and transmitting the ingredient information periodically or in response to events to the server. The server stores the ingredient information in the database, including fields for ingredient type, quantity, unit, and expiration date.

The server represents the condition information and the ingredient information as in-memory data structures, for example, key-value maps, lists, and records. The server normalizes these structures by mapping user-entered labels to canonical feature names and by aggregating ingredient quantities by type. The server then generates a prompt sentence by concatenating textual representations of the features in a predetermined template. For example, the server generates a prompt sentence such as:
“User conditions: healthy, for children, deliverable within 15 minutes. Current refrigerator ingredients: chicken breast 300 g, carrots 2, milk 500 ml, eggs 4. Please generate three dinner menu options that maximize the use of the listed ingredients.”

In another example, the server generates a prompt sentence such as:
“User conditions: diet type=low-carb, target audience=family with children, max delivery time=20 minutes. Refrigerator contents: chicken breast 400 g, lettuce 1 head, tomatoes 3, yogurt 200 g. Please generate three dinner menu options using as many refrigerator ingredients as possible. For each option, output a menu title, a short description, a list of required ingredients with quantities, and a list of missing ingredients.”

The server supplies the prompt sentence to the generative AI model via an application programming interface. The generative AI model is implemented as a deep neural network with multiple layers, including at least an input embedding layer, a plurality of transformer blocks, and an output projection layer. The server encodes the prompt sentence as a sequence of token identifiers and passes it along with control parameters such as maximum output length and sampling temperature. The generative AI model processes the token sequence using parallel matrix multiplications and non-linear activation functions on dedicated computation hardware, and outputs a sequence of tokens that the server decodes to menu information in natural language form.

The server implements a menu analysis unit using parsing algorithms that operate on the generated menu information. The server applies heuristic pattern matching, tokenization, and rule-based segmentation to identify menu titles, descriptions, and required ingredient entries in the generated text. The server converts the parsed information into a structured representation, such as a list of menu objects, each containing fields for menu title, description, and a list of required ingredient items with associated quantities and units. This conversion reduces ambiguity inherent in natural language output and allows subsequent algorithmic comparison with the ingredient information.

The server then executes a shortage information calculation unit. The server iterates through each required ingredient list and compares required quantities with available quantities from the storage apparatus. The server performs arithmetic subtraction and threshold comparison for each item, and when the available quantity is lower than the required quantity, the server records the difference as shortage ingredient information. The server may maintain these comparisons in dedicated data structures, such as hash maps keyed by ingredient type, to achieve constant-time lookup and reduce computation time compared with naive linear scans.

The server computes shortage ingredient information in this manner for each generated menu, and then integrates the shortage ingredient information back into the menu objects. The server generates a final representation of menu information that includes, for each menu, a required ingredient list and a shortage ingredient list. The server sends this integrated menu information to the terminal. The terminal displays the menu information to the user, highlighting which ingredients are available and which are missing, and showing relevant metadata such as estimated preparation time and suitability for the specified health state. The user selects a preferred menu and confirms an order from the terminal interface. The terminal sends menu selection information and order confirmation information to the server. The server constructs order information including at least a selected menu identifier, user identification information, delivery destination, and service time constraint. The server stores this order information in the database and also transmits the order information to a delivery service device via a network interface.

In some embodiments, the server further processes expiration date information and operation information of the storage apparatus. The server evaluates a cost function that balances multiple objectives, including maximizing use of ingredients that are close to expiration, minimizing the number of additional ingredients to be delivered, and reducing power consumption of the storage apparatus. For example, the server assigns higher weights to ingredients with earlier expiration dates and configures the generative AI model input to emphasize those ingredients in the prompt sentence. The server also selects menus that can be prepared without significantly altering the operation mode of the storage apparatus, thereby reducing the frequency of door openings and compressor cycles. By encoding these constraints and preferences as structured features within the prompt sentence and within the subsequent decision logic, the server optimizes both food utilization and energy usage.

The server improves computer technology in several ways. The server optimizes the prompt sentence generation so that the generative AI model receives a compact and structured representation of relevant features, reducing the number of tokens and therefore the computation time required for inference. This reduction in token length and redundancy leads to lower latency for menu generation and reduced network bandwidth usage. The server further reduces downstream processing costs by translating unstructured model output into structured menu objects immediately after generation, allowing subsequent algorithms to operate on fixed-format data instead of performing repeated text parsing.

The server implements a model architecture and training procedure that are tailored to the menu generation task. The generative AI model is pre-trained on large-scale text data and then fine-tuned on domain-specific corpora containing recipes, nutrition information, and delivery constraints. During fine-tuning, the server or an associated training module uses a loss function that combines language modeling error with additional penalty terms related to ingredient consistency and time constraint satisfaction. The model updates its weights via gradient descent with backpropagation, using mini-batch optimization and regularization methods. Data augmentation may be applied to training data, such as random substitution of ingredients within the same category and random permutation of constraint expressions, to improve robustness of the model to variations in prompt sentence structure.

The server uses non-conventional processing rules that differ from simple human heuristics. The server applies ranking algorithms that score candidate menus not only on user preference features but also on energy usage profiles and projected food waste reduction. The server computes these scores based on numerical features derived from expiration date distributions and operation history of the storage apparatus. The server then instructs the generative AI model via specialized prompt patterns that encode these scores as guidance tokens, steering inference in a way that is difficult or impractical to replicate manually. This cooperation between structured scoring and generative inference enables the system to search a larger solution space with higher computational efficiency than human planners or rule-based systems.

The server also improves data management by maintaining a consistent schema across user condition information, ingredient information, menu information, and order information. The server defines normalized tables or collections with indices on fields such as ingredient type, expiration date, and user identifier. These indices enable fast retrieval of relevant records when constructing prompt sentences and when computing shortage ingredient information.

The server reduces communication load between components by caching frequently used ingredient patterns and menu templates; when the server detects that new conditions are similar to previously processed conditions, the server can partially reuse prompt sentence fragments and previously computed shortage ingredient profiles, thereby minimizing redundant computation and data transmission.

The terminal and the server together achieve technical effects beyond mere automation of human mental tasks. The server coordinates sensor readings from the storage apparatus, high-dimensional neural network computations, structured comparison operations, and network-based order transmission. By designing specific data structures and processing flows, the server attains improved throughput, lower latency, and more accurate matching between generated menus and real-world constraints. The server reduces error rates in ingredient matching by using automated consistency checks between generated required ingredient lists and known inventory data, and if inconsistencies exceed a threshold, the server regenerates or adjusts the prompt sentence to refine the generative AI model output.

Alternative embodiments are possible. In one variation, the server deploys the generative AI model locally on a dedicated accelerator board rather than accessing it as a remote service. In another variation, the server employs a different neural network architecture, such as a recurrent neural network or a hybrid architecture combining convolutional and transformer layers, while still using structured prompt sentences as input. In yet another variation, the server uses multiple generative AI models, each specialized for a different cuisine type or diet type, and selects or ensembles outputs from these models based on the user's condition information and the contents of the storage apparatus.

In another embodiment, the server executes a rule-based pre-filtering stage before invoking the generative AI model. The server eliminates infeasible ingredient combinations based on allergen information or strong dietary restrictions, and encodes the remaining combinations into the prompt sentence. This reduces the search space that the generative AI model must explore and increases the probability that the first generated menus will satisfy constraints, thereby improving computational efficiency and user experience.

The server is therefore configured not only to perform high-level business logic but also to execute specific low-level computational processes that transform raw sensor data and user input into optimized prompt sentences, that drive a sophisticated generative AI model, and that convert its output into actionable, resource-aware order information. This configuration yields concrete technical advantages, including faster response times, reduced network usage, improved accuracy of ingredient and constraint handling, and decreased power consumption and food waste in the physical storage apparatus coupled to the system.

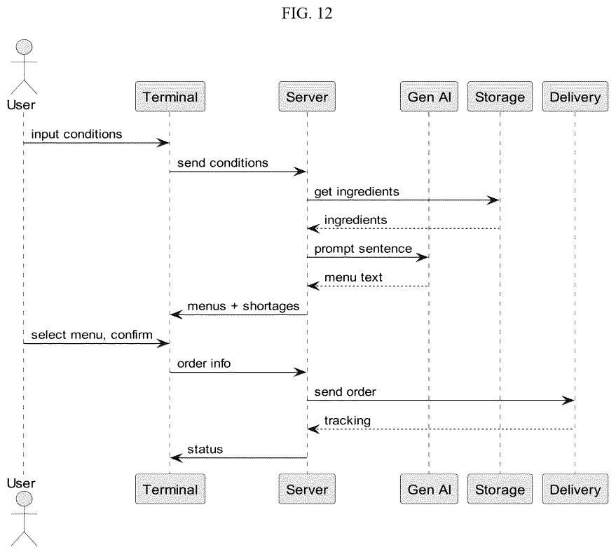

The following describes the processing flow using FIG. 12.

### Step 1

The user operates the terminal to input condition information. The terminal displays input fields for health state, target audience, and service time constraints, and the user enters data such as “healthy,” “for children,” and “deliverable within 15 minutes.” The input of this step is raw user keystrokes and selections on the user interface. The terminal converts these UI events into structured condition data, for example, internal key-value pairs, and validates required fields and value formats. The output of this step is validated condition information stored in the terminal's memory.

### Step 2

The terminal transmits the validated condition information to the server. The input of this step is the structured condition data produced in Step 1. The terminal serializes the condition information, encapsulates it in a network message, and sends it to an API endpoint on the server via a secure communication protocol. The output of this step is an incoming request message received by the server that contains the user's condition information.

### Step 3

The server receives and parses the condition information. The input of this step is the network request containing the serialized condition data. The server decodes the network message, parses the payload using a parser, and maps the parsed data into in-memory structures, such as a condition object with fields for health state, subject attributes, and service time constraints. The output of this step is a normalized condition object held in the server's working memory.

### Step 4

The server acquires ingredient information from the storage apparatus. The input of this step is a storage identifier associated with the user or previously stored linkage information. The server issues a query or a request to the storage apparatus or to a database that stores sensor-derived ingredient information, and receives records including ingredient type, quantity, unit, and expiration date. The server aggregates quantities for identical ingredient types and normalizes units when necessary. The output of this step is an ingredient information data structure representing current contents of the storage apparatus.

### Step 5

The server generates a prompt sentence for a generative AI model. The inputs of this step are the normalized condition object from Step 3 and the ingredient information from Step 4. The server performs data concatenation and string formatting: it converts each field (for example, health state, target audience, max delivery time, ingredient types and quantities) into textual fragments and arranges them in a predefined template. As a result, the server creates a prompt sentence such as “User conditions: healthy, for children, deliverable within 15 minutes. Current refrigerator ingredients: chicken breast 300 g, carrots 2, milk 500 ml, eggs 4. Please generate three dinner menu options that maximize the use of the listed ingredients.” The output of this step is a complete prompt sentence suitable for input to the generative AI model.

### Step 6

The server invokes the generative AI model with the prompt sentence. The input of this step is the prompt sentence generated in Step 5 together with control parameters such as maximum output length and sampling temperature. The server tokenizes the prompt sentence into a sequence of token identifiers, packages the token sequence and parameters into a model request, and sends it to the generative AI model's inference interface. The generative AI model executes internal neural network computations, including matrix multiplications and non-linear activations, to predict subsequent tokens. The output of this step is a generated token sequence returned to the server, which the server decodes into natural language menu information.

### Step 7

The server analyzes the generated menu information. The input of this step is the natural language menu text obtained in Step 6. The server performs data processing that includes tokenization, sentence segmentation, and rule-based pattern matching to identify menu titles, descriptions, and lines containing ingredients and quantities. The server converts these elements into structured menu objects, each containing a title field, a description field, and a list of required ingredient items with type and quantity. The output of this step is a collection of structured menu objects in the server's memory.

### Step 8

The server calculates shortage ingredient information by comparing required ingredients with available ingredients. The inputs of this step are the structured menu objects from Step 7 and the ingredient information data structure from Step 4. The server iterates over each required ingredient in each menu, performs a lookup in the ingredient information data structure, and computes a difference between required quantity and available quantity using arithmetic subtraction. When the required quantity exceeds the available quantity, the server records the deficit as shortage ingredient information. The output of this step is, for each menu object, a shortage ingredient list that specifies which items and what amounts are missing.

### Step 9

The server integrates shortage ingredient information into the menu information and prepares a response for the terminal. The inputs of this step are the menu objects produced in Step 7 and the shortage ingredient lists obtained in Step 8. The server attaches the corresponding shortage list to each menu object, optionally computes cost estimates or other metrics using arithmetic operations and data lookups, and serializes the enriched menu objects into a response format. The output of this step is a response payload containing menu titles, descriptions, required ingredients, shortage ingredients, and any additional metadata.

### Step 10

The terminal receives and displays the menu information to the user. The input of this step is the response payload sent from the server in Step 9. The terminal deserializes the payload into internal structures, such as menu view models, and performs data binding to graphical components. The terminal renders lists or cards that show menu titles, summaries, and visual indicators for missing ingredients and preparation or delivery times. The output of this step is a rendered user interface that allows the user to inspect and compare proposed menus.

### Step 11

The user selects a menu and confirms an order. The input of this step is the displayed menu list on the terminal screen. The user taps or clicks on a desired menu, optionally specifies a number of servings or other options, and activates an order confirmation control. The terminal captures these interactions and aggregates them into menu selection information and order confirmation information. The output of this step is structured order input data retained in the terminal's memory.

### Step 12

The terminal sends the order confirmation information to the server. The input of this step is the structured order input data produced in Step 11. The terminal serializes the order information, including selected menu identifier, quantity, user identification, and delivery address, into a network message and transmits it via a secure protocol to the server's order reception endpoint. The output of this step is an incoming order request received by the server.

### Step 13

The server validates and stores the order information. The input of this step is the order request message arriving from the terminal in Step 12. The server parses the message, checks data types, verifies that the referenced menu exists and that mandatory fields such as address and service time are present, and rejects or corrects inconsistent values. The server then writes the validated order information into persistent storage by executing database insert operations or equivalent data storage operations. The output of this step is a stored order record with a unique order identifier in the server's storage device.

### Step 14

The server transmits order information to a delivery service device. The input of this step is the stored order record from Step 13. The server extracts delivery-related fields, such as destination, ordered items, and requested time window, formats these fields into a delivery request structure, and sends the structure to a delivery service device via a network interface. The server may receive a tracking identifier and estimated delivery time in response and associate them with the order record by updating the relevant database entries. The output of this step is a confirmed delivery request with tracking information stored in the server's system.

### Step 15

The terminal provides order status to the user. The inputs of this step are the tracking information and status updates stored and managed by the server in Step 14 and subsequent status changes. The terminal periodically requests status information from the server or receives push notifications, parses the received status data, and updates on-screen indicators such as “Order confirmed,” “Out for delivery,” and “Delivered.” The output of this step is a dynamic display on the terminal that reflects the current state of the order for the user.

It is also possible to incorporate an emotion engine for estimating the user's emotions. That is, the specific processing unit 290 may estimate the user's emotions using an emotion identification model 59, and perform specific processing based on the estimated emotions.

### Example 2

Description follows regarding a flow of the specific processing in an Example 2. The units of the system described below are implemented by the data processing device 12 and the smart device 14. The data processing device 12 is called a “server” and the smart device 14 is called a “terminal”.

Conventional inventory management and recommendation systems that operate with storage apparatuses, such as household refrigeration units, typically rely on manually entered item lists or simple barcode scanning. Such systems suffer from several technical limitations in terms of computer technology. First, they do not robustly integrate heterogeneous sensor inputs, such as image data from imaging devices and physical quantity data from detection devices, into a unified, machine-interpretable inventory representation. As a result, processors in such systems cannot reliably infer item types, quantities, and states, which leads to inaccurate or stale inventory data and increases computational overhead due to repeated manual correction and redundant processing.

Second, known systems generally treat interaction with generative AI models as a simple text-forwarding operation: the system passes user text to a remote model and returns the model's output without deep integration with structured inventory data or user constraints. This naive coupling between backend inventory data and generative output causes several computational inefficiencies. For example, a processor must either pre-process large volumes of data for every request or the generative model must internally re-infer constraints from loosely formatted text, resulting in increased processing time, increased network traffic, and inconsistent outputs that do not accurately reflect the actual state of the storage apparatus.

Third, existing solutions do not implement a well-defined, machine-optimized mechanism for programmatically constructing prompt sentences as instruction sentences to generative AI models. Without system-level logic that systematically fuses inventory information, user conditions, and system policies into a parameterized prompt, it is difficult for the processor to generate reproducible and controllable outputs from a generative AI model. This lack of structure degrades the quality of the generated proposals and prevents efficient reuse of intermediate computational results (such as consolidated inventory states) across multiple user queries.

Fourth, conventional systems rarely perform automated analysis of generated proposal information against current inventory data to identify item shortages and candidate substitutions in a structured manner. In many cases, a user must manually compare generated recipes or plans with the inventory, which fails to exploit the computational capabilities of the processor and results in a fragmented processing pipeline. This not only imposes additional cognitive and operational load on the user, but also increases the likelihood of underutilization or waste of perishable items and inefficient usage of storage resources.

Fifth, while some systems consider preferences such as “healthy” or “low-cost,” they do not tightly integrate user health-related conditions, use-by date information, and storage condition information at the processor level when constructing prompts and interpreting generative outputs. This omission means that the computer system does not automatically optimize proposal generation to reduce discarded food and optimize energy consumption of the storage apparatus. Consequently, processors in such systems cannot fully leverage available sensor and profile data to perform context-aware control of generative AI interactions, leading to suboptimal resource usage and limited technical effect in terms of system efficiency and responsiveness.

Accordingly, there is a need for an improved computer-implemented system and processing method in which a processor (i) automatically consolidates sensor-derived inventory information and transaction-derived inventory information into a unified data structure, (ii) programmatically constructs prompt sentences for a generative AI model based on that unified inventory information and user conditions, and (iii) post-processes the generative output to compute shortage and substitution information. Such a system should technically improve the way computers manage heterogeneous data flows and control generative AI models, thereby reducing redundant computations, improving data consistency, and enabling more effective and efficient utilization of storage apparatuses and computational resources.

The specific processing by the specific processing unit 290 of the data processing device 12 in Example 2 is realized by the following means.

The present invention provides a server comprising a processor configured to acquire use conditions from a user through an information input/output device and store the use conditions in association with the user, acquire image data and physical quantity data from an imaging device and a detection device disposed in a storage apparatus, execute an image processing program on the image data to recognize a type of an item contained in the storage apparatus, estimate a quantity and a state of the item on the basis of the physical quantity data, and integrate a recognition result and an estimation result to generate inventory information, analyze a transaction record image acquired by the user using a character recognition program to extract purchased item information from a text recognition result and update the inventory information on the basis of the purchased item information, generate a prompt sentence as an instruction sentence for input to a generative information processing model on the basis of the inventory information and the use conditions and input the prompt sentence into the generative information processing model as a prompt to cause the generative information processing model to generate proposal information corresponding to the inventory information and the use conditions, compare required items included in the proposal information with items included in the inventory information to identify insufficient items and substitutable items and generate output data by adding a result of the identification to the proposal information, and transmit the output data to the information input/output device to present to the user the proposal information including information on the insufficient items and the substitutable items. This enables a computer-implemented system to automatically consolidate heterogeneous sensor data and transaction data into structured inventory information, to programmatically construct and control prompt sentences for a generative AI model based on that structured information and user conditions, and to post-process generative outputs to compute and present shortage and substitution information, thereby improving the technical efficiency, accuracy, and responsiveness of inventory management and proposal generation performed by the processor.

The term “processor” refers to a hardware information processing unit, such as a central processing unit or a microcontroller, that executes instructions of a program to perform data acquisition, analysis, generation, and control operations in the system.

The term “information input/output device” refers to an electronic user interface apparatus, such as a terminal including a display and an input unit, that enables a user to input conditions and to receive and present output data from the server.

The term “use conditions” refers to information specifying constraints, preferences, or requirements of a user, including at least one condition related to utilization of stored items, and optionally including conditions related to health, cost, preparation time, or other usage parameters.

The term “storage apparatus” refers to an equipment unit configured to store items under controlled environmental conditions, such as a refrigeration unit, a freezing unit, or another temperature-controlled storage device.

The term “imaging device” refers to an image acquisition element, such as an electronic image sensor or camera module, that captures image data representing an interior region of the storage apparatus.

The term “detection device” refers to a sensor element, such as a weight sensor, temperature sensor, or other physical quantity sensor, that outputs physical quantity data indicative of at least one characteristic of items or an environment within the storage apparatus.

The term “image data” refers to digital data representing at least one image captured by the imaging device, including pixel values that can be processed by an image processing program to recognize items.

The term “physical quantity data” refers to digital measurement data output from the detection device, including at least one physical parameter such as weight, temperature, humidity, or door-open state.

The term “image processing program” refers to a software component executed by the processor that processes image data to detect and recognize objects, including operations such as pre-processing, segmentation, object detection, and classification.

The term “character recognition program” refers to a software component executed by the processor that analyzes an image containing textual content and outputs a text recognition result, for example by optical character recognition processing.

The term “item” refers to a stored object or article, such as a food product or ingredient, that is placed inside the storage apparatus and is subject to recognition, quantity estimation, and state estimation by the system.

The term “quantity” refers to an estimated amount or count of an item, which may be expressed in units such as mass, volume, or piece count and is derived from physical quantity data and inventory records.

The term “state” refers to an estimated condition of an item, including at least one of freshness, remaining quantity level, or appropriateness of storage conditions, inferred from physical quantity data, time information, and environmental conditions.

The term “inventory information” refers to structured data generated and maintained by the processor that represents types, quantities, locations, time-related information, and states of items stored in the storage apparatus for a given user.

The term “transaction record image” refers to image data representing a record of a commercial transaction, such as a receipt or invoice, which contains information about purchased items and is captured by a user.

The term “purchased item information” refers to data extracted from a transaction record image, including at least one of an identifier of a purchased item, a quantity of the purchased item, and a purchase date, used to update the inventory information.

The term “generative information processing model” refers to a machine-implemented model, such as a generative AI model or a language model, that generates output information, including text, based on an input instruction sentence and context data.

The term “prompt sentence” refers to an instruction sentence generated by the processor, including embedded inventory information and use conditions, that is supplied as an input to the generative information processing model to control generation of proposal information.

The term “proposal information” refers to information generated by the generative information processing model in response to the prompt sentence, including at least one proposal related to utilization of items, such as a recipe, plan, or recommendation.

The term “required items” refers to items that are indicated in the proposal information as necessary for executing a proposed action, such as preparing a recipe or following a plan, and that are subject to comparison with items recorded in the inventory information.

The term “insufficient items” refers to required items for which an available quantity recorded in the inventory information is missing or smaller than a quantity indicated by the proposal information.

The term “substitutable items” refers to items that are present in the inventory information and are determined by the processor to be usable in place of or in combination with insufficient items according to predefined or learned substitution rules.

The term “output data” refers to data generated by the processor that includes at least the proposal information and additional information about insufficient items and substitutable items, and that is transmitted to the information input/output device for presentation.

The term “health-conscious proposal information” refers to proposal information that is generated by the generative information processing model in consideration of at least one health-related use condition of the user, such as dietary restrictions or nutritional goals.

The term “use-by date information” refers to time-related data associated with an item in the inventory information, indicating a recommended latest date for use or consumption of the item.

The term “storage condition information” refers to data describing environmental or operational conditions under which an item is stored in the storage apparatus, including at least one of temperature, humidity, and compartment type.

The term “economic benefit” refers to a beneficial effect achieved for the user by operation of the system, including at least one of reduction in discarded items, reduction in energy consumption, and improvement in cost-effective utilization of purchased items.

In one embodiment, a server cooperates with a terminal and a user to manage items stored in a storage apparatus and to generate proposals using a generative AI model. The server includes at least one processor, a memory, a network interface, and a non-volatile storage device. The terminal includes a display, an input unit, and, in some embodiments, an imaging unit. The storage apparatus includes at least one imaging device and at least one detection device, such as a weight sensor or a temperature sensor.

The server executes an operating system such as a general-purpose server operating system and runs application software including an image processing program, a character recognition program, an inventory management module, a prompt construction module, and a generative AI interface module. In one embodiment, the server executes the image processing program using a computer vision library such as an implementation of OpenCV, executes a neural-network-based object detector implemented using a deep learning framework such as a TensorFlow-compatible framework or a PyTorch-compatible framework, and executes the character recognition program using an optical character recognition engine such as a Tesseract-compatible engine. The server communicates with the generative AI model using a network-based API or using a local inference engine implemented, for example, with a transformer-based language model library.

The terminal executes an application that presents a graphical user interface. The terminal may be implemented as a smartphone, a tablet, or a general-purpose computing device. The terminal provides an input screen on which the user inputs use conditions, such as dietary restrictions, time constraints, and desired number of servings. The terminal also provides an input field for a free-form prompt sentence to be used as an instruction to the generative AI model. For example, the user may input one of the following prompt sentences:
“Suggest a high-protein dinner for two people using what I have in my fridge.”
“Using only what is currently in my fridge and pantry, generate three quick dinner ideas for one person.”
“Generate a 3-day low-carb meal plan for two adults, using as many existing ingredients as possible and minimizing food waste.”
“Based on my current inventory, propose a vegetarian lunch that takes less than 20 minutes to prepare.”

The server stores the use conditions and the prompt sentence in a structured data format in the memory. In one embodiment, the server uses a relational database management system to store the use conditions in a user profile table and to associate the use conditions with user identifiers. The server uses explicit fields such as “diet_type,” “allergy_list,” “max_cook_time,” and “budget_level” so that the processor can later retrieve these values without parsing free-form text.

The server acquires image data and physical quantity data from the storage apparatus. The server receives image frames captured by the imaging device in the storage apparatus, for example a digital camera mounted inside the refrigerator compartment. The server also receives weight readings from a set of weight sensors installed on shelves and, optionally, temperature readings from temperature sensors in each compartment. The server stores raw images in a file storage system and stores physical quantity data in time-stamped records in a sensor table.

The server executes the image processing program to transform the raw image data into a normalized representation suitable for object detection. The server performs operations such as resizing images to a fixed resolution, converting color spaces, normalizing pixel intensities, and applying noise reduction filters. The server then executes a convolutional neural network-based object detection model that has been trained in advance. In one embodiment, the server uses a model architecture in which convolutional layers extract hierarchical visual features, feature maps are passed through a region proposal module, and a classifier head assigns class probabilities to each region. The server uses bounding box regression to compute spatial coordinates of detected items.

The server uses the output of the object detection model to generate intermediate item candidates. Each candidate includes at least an item type label, a confidence score, and spatial coordinates. The server filters candidate items using a confidence threshold and non-maximum suppression, thereby reducing redundant detections. This structured detection pipeline improves processing speed and reduces memory usage compared with naive per-pixel or template-matching approaches, because the server operates only on a small set of bounding boxes rather than on entire images at later stages.

The server uses the physical quantity data to estimate quantities and states of items. The server associates weight sensor readings with shelves or regions corresponding to the detected items. The server maintains, in a database, baseline weight values recorded when new items are added and uses these baseline values together with current weight readings to estimate remaining quantities. For example, for a liquid container, the server computes remaining volume by subtracting current weight from baseline weight and dividing by density. For packaged items such as eggs or bottles, the server uses known per-unit weights stored in a product catalog table. The server also evaluates temperature readings to determine whether current storage conditions meet recommended ranges stored in the catalog table. The server integrates these estimations with the visual recognition results to create inventory records with item identifiers, estimated quantities, and state flags such as “low quantity” or “improper temperature.”

The server further uses a character recognition program to integrate transaction data into the inventory information. The user operates the terminal to capture an image of a transaction record, such as a shopping receipt. The terminal sends the transaction record image to the server. The server converts the transaction record image into grayscale, applies binarization and deskewing, and then executes an optical character recognition algorithm. The character recognition program outputs a text string representing the receipt contents. The server parses the text string, detects patterns corresponding to product names, quantities, and prices, and maps product names to canonical product identifiers using a dictionary table and, optionally, a fuzzy matching algorithm. The server then updates the inventory records by adding or adjusting quantities based on the recognized purchased items. This automatic integration of sensor-based inventory data and transaction-based inventory data improves the completeness and accuracy of the inventory information while reducing manual input.

The server consolidates the inventory information into a unified data structure. The server uses an inventory table in which each record represents a specific item for a specific user and includes fields such as item identifier, location identifier, current quantity, estimated use-by date, and storage condition status. The server resolves inconsistencies by applying rule-based logic. For example, when the server detects a new item visually but no corresponding weight change or receipt record is found, the server maintains a lower confidence level for that entry and marks it for re-verification in a subsequent image capture. Conversely, when both visual detection and transaction recognition agree on an item, the server increases a confidence value and uses that value in subsequent processing. By maintaining such confidence values and consistency checks, the server reduces false positives and improves robustness of the inventory data compared with systems that simply overwrite records.

The server constructs a prompt sentence for the generative AI model in a structured manner using the unified inventory information and the stored use conditions. Instead of passing only the user's raw text prompt, the server generates an extended instruction sentence that encodes the inventory, user constraints, and system policies in a predictable format. For example, if the user inputs “Suggest a high-protein dinner for two people using what I have in my fridge,” the server may internally construct the following extended prompt sentence: “Inventory: chicken breast 300 g, broccoli 1 head, carrots 2, eggs 4, plain yogurt 200 g, rice 300 g, olive oil, salt, pepper. User conditions: high-protein, low-fat, two servings, max cooking time 30 minutes, no peanuts. Task: Based on the above inventory and user conditions, suggest three detailed dinner recipes that are high in protein and mainly use the listed ingredients.”

The server generates this extended prompt sentence by programmatically enumerating items from the inventory table, filtering items based on their states (for example, prioritizing items close to their use-by date), and embedding explicit parameters such as maximum cooking time and number of servings. The server uses a deterministic formatting scheme so that the generative AI model receives inputs with consistent structure. This structured prompt construction improves reproducibility of the generative outputs and reduces the amount of context that the model must infer implicitly, thereby reducing token usage and lowering network and computational load.

The server interfaces with a generative AI model that may be implemented as a transformer-based language model. In one embodiment, the generative AI model is a multi-layer neural network comprising an embedding layer, multiple self-attention layers, and a feed-forward network for each layer. The generative AI model has been trained on a large corpus of text data including recipes and general language. During training, the model uses a loss function such as cross-entropy to minimize prediction errors between generated tokens and ground-truth tokens. The model updates weight parameters using an optimization algorithm such as gradient descent with adaptive learning rate. The training process may include techniques such as mini-batch training, dropout, layer normalization, and data augmentation by text masking or paraphrasing.

The server uses the generative AI model in an inference phase. The server sends the extended prompt sentence as a sequence of tokens to the model, optionally together with control parameters such as temperature, top-k value, and maximum token length. The generative AI model computes attention scores for each token, propagates representations through transformer layers, and outputs probability distributions over possible next tokens. The server uses a sampling or decoding strategy such as greedy decoding or nucleus sampling to generate a complete sequence representing proposal information, such as one or more recipes or a multi-day meal plan.

The server performs post-processing of the generated text in a manner that improves technical performance. The server parses the generated text to extract structured information such as ingredient lists and required quantities. The server uses pattern-matching rules and, optionally, a lightweight parsing model to identify lines that correspond to ingredients and steps. The server then compares the list of required items with the inventory table. For each required item, the server normalizes the item name and looks up matching inventory records.

The server converts required units (for example, cups or tablespoons) into base units such as grams or milliliters using conversion tables. The server subtracts required quantities from available quantities in the inventory table to determine whether the item is sufficient or insufficient. When an item is insufficient, the server records the shortfall and searches for substitutable items using a substitution rules table. The substitution rules table may include pairs such as “lemon juice” and “lime juice” or “butter” and “olive oil,” associated with similarity scores and constraints. The server therefore generates a shortage list and a substitution suggestion list.

The server generates output data that includes both the proposal information and the shortage and substitution information. The server structures this data so that the terminal can display recipes, missing items, and suggested substitutes in separate sections. The server sends the output data to the terminal via a network protocol. The terminal receives the output data, updates the graphical user interface, and presents the proposals to the user along with visual indicators for missing items.

The server thereby achieves technical improvements in several respects. Because the server constructs prompt sentences using structured inventory information and use conditions, the generative AI model receives more precise and compact context. This reduces unnecessary re-computation of constraints inside the model and decreases the number of tokens transmitted and processed, which results in lower latency and reduced communication bandwidth. Because the server maintains unified inventory information that merges sensor data and transaction data, the server can avoid repeated manual corrections and redundant database writes, improving consistency and reducing storage overhead. Because the server post-processes generative outputs to compute shortages and substitutions programmatically, the server offloads complex comparison and reasoning tasks from the user, and performs them in a standardized, machine-efficient way.

The server also improves computational accuracy and stability by using explicit confidence values, baseline weights, and rule-based consolidation of visual and transactional data. For instance, by associating each inventory entry with a confidence score that depends on the agreement between imaging results and transaction results, the server can selectively prioritize high-confidence data in prompt construction. This improves the relevance of the generated proposals and reduces the probability that the generative AI model will rely on outdated or incorrect inventory information.

The generative AI model operates with internal mechanisms different from straightforward human reasoning. For example, the model uses high-dimensional vector representations of tokens and multi-head attention mechanisms to capture relationships between ingredients, cooking methods, and constraints. These internal representations are not simple rule lists but learned statistical structures that capture co-occurrence patterns, substitution relations, and typical recipe formats. The server exploits these properties by providing structured prompts that align with the model's learned representation space, thereby enhancing its ability to produce coherent and constraint-respecting outputs.

The server further improves computer technology by organizing the overall inventory and generation pipeline into modular components with well-defined data structures. The server uses separate tables or data structures for raw sensor data, processed item candidates, transaction-derived purchases, consolidated inventory, user profiles, and generative outputs.

By separating concerns in this way, the server minimizes contention between database operations and reduces the need for complex joins across unstructured data. This modular architecture facilitates incremental updates, such as recalculating quantities only for shelves where the weight has changed, which in turn reduces processor load and energy consumption.

The system is not limited to a particular deployment mode. In one variation, the server executes the generative AI model locally on a specialized hardware accelerator such as a graphics processing device or a tensor processing device, thereby reducing network latency and improving privacy. In another variation, the server communicates with an external model via a remote application programming interface and applies additional caching logic to reuse previous generative outputs when inventory and use conditions have not significantly changed. The server can also adjust model parameters such as decoding temperature based on the confidence in inventory data, for example using lower temperature when inventory data is precise to obtain more deterministic outputs.

The system can be applied to various storage apparatuses beyond household refrigerators. For example, the storage apparatus can be a freezer, a pantry with ambient sensors, or an industrial cold storage unit. In each case, the imaging device and detection device may be adapted to the environment, and the inventory information schema may be extended to include additional attributes such as batch numbers or quality inspection results. The core mechanisms of image-based item recognition, sensor-based quantity and state estimation, transaction-based inventory updating, structured prompt sentence construction, and post-processing of generative outputs remain applicable and provide similar technical benefits.

The server, the terminal, and the user therefore cooperate to implement an integrated, computer-implemented system that improves how computers manage heterogeneous sensory and transactional data, how they construct and control prompt sentences to a generative AI model, and how they interpret model outputs to deliver technically grounded proposals. By tightly coupling the generative AI model with structured, sensor-derived inventory data and explicit use conditions, the system goes beyond mere automation of human planning and instead enhances the underlying computer technology for data fusion, model control, and resource-aware decision support.

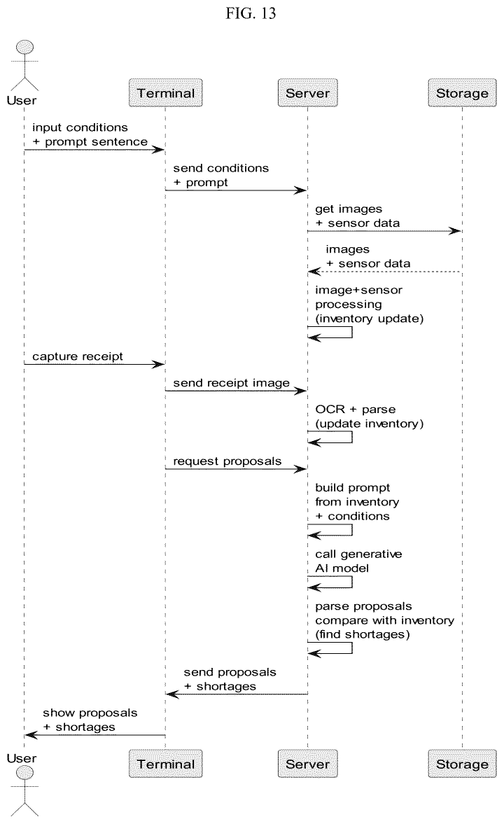

The following describes the processing flow using FIG. 13.

### Step 1

Server initializes system modules and data structures.
Server loads configuration parameters from persistent storage, including sensor mapping information, product catalog definitions, substitution rules, and model interface settings. As input, server reads configuration files and database records. Server instantiates software modules such as an image processing module, a character recognition module, an inventory management module, a prompt construction module, and a generative AI interface module.
As output, server produces initialized in-memory data structures, such as sensor-to-shelf mapping tables, product identifier dictionaries, and empty caches for inventory snapshots, which are used in subsequent steps.

### Step 2

Server acquires sensor data from the storage apparatus.
Server receives, as input, image data from an imaging device and physical quantity data from detection devices in the storage apparatus via a communication interface. Server records a timestamp, associates each incoming data packet with a user identifier and a storage location identifier, and stores the raw image files in object storage and the raw sensor values in a sensor table. Server applies basic validation checks to confirm that each data sample falls within allowed ranges. As output, server produces validated sensor records and stored images referenced by unique identifiers.

### Step 3

Server performs image pre-processing on captured images.
Server retrieves, as input, raw image files associated with the latest capture event. Server performs image resizing to a fixed resolution, converts color space (for example, from BGR to RGB), normalizes pixel intensities to a standard range, and applies noise reduction filters using an image processing program. Server optionally corrects lens distortion and adjusts brightness and contrast. As output, server generates pre-processed image tensors or matrices that are suitable as input to a convolutional neural network.

### Step 4

Server executes object detection to recognize item types.
Server feeds, as input, the pre-processed image tensors into a trained object detection neural network. Server performs convolution operations, pooling, feature extraction, and bounding box regression according to the model architecture, and the network outputs candidate bounding boxes with class probabilities and confidence scores. Server filters out low-confidence detections and applies non-maximum suppression to merge overlapping boxes. As output, server produces a list of detected item candidates, each including an item type label, a confidence value, and bounding box coordinates.

### Step 5

Server estimates item quantities and states from physical quantity data.
Server reads, as input, weight readings and temperature readings associated with each shelf or compartment from the sensor table. Server aggregates readings over a short time window to reduce noise, then computes derived values such as average weight per shelf and average temperature per zone. Server compares current weights with stored baseline weights for known items, calculates remaining quantities through subtraction and unit conversion, and evaluates temperature values against recommended ranges in the product catalog. As output, server updates internal records for each item candidate with estimated quantities and state flags, such as “low quantity,” “near expiration,” or “improper temperature.”

### Step 6

Server integrates visual detections and sensor estimations into inventory records.
Server takes, as input, the list of item candidates from object detection and the quantity and state estimations from sensor analysis. Server matches each detected item candidate to a shelf region, applies rules to associate weight changes with specific items, and merges this information with existing entries in an inventory table. Server computes a confidence score for each inventory record based on agreement between visual detection, weight change, and historical data. As output, server generates updated inventory records containing item identifiers, locations, quantities, states, and confidence values, and stores them in the inventory table.

### Step 7

User captures a transaction record image using the terminal.
User operates the terminal to open a receipt capture function and points the terminal's imaging unit at a physical receipt. Terminal receives, as input, light reflected from the receipt and converts it into a digital image. Terminal may perform local pre-processing, such as cropping, rotation correction, and compression. As output, terminal produces a transaction record image file with associated metadata, such as capture time and user identifier.

### Step 8

Terminal transmits the transaction record image to the server.
Terminal takes, as input, the transaction record image file and user authentication information. Terminal packages these into a network request and sends the request via a secure protocol to the server. As output, terminal produces a data packet containing the image and identification headers, and server receives this packet as input for further processing.

### Step 9

Server executes character recognition on the transaction record image.
Server obtains, as input, the transaction record image from the terminal. Server converts the image to grayscale, applies binarization, deskewing, and noise reduction using the image processing program, and then passes the processed image to the character recognition program. The character recognition program performs segmentation into text lines and characters and computes character probabilities to generate a recognized text string. As output, server produces a plain-text representation of the transaction record.

### Step 10

Server parses recognized text to extract purchased item information.
Server uses, as input, the recognized text string from character recognition. Server splits the text into lines, applies regular expressions and parsing rules to identify product name fields, quantity fields, and price fields, and normalizes product names (for example, by lowercasing and trimming). Server then maps normalized product names to canonical product identifiers stored in a product catalog table, optionally using approximate string matching when exact matches are not found. As output, server produces a structured list of purchased items, each including a product identifier, quantity, and purchase date.

### Step 11

Server updates inventory information using purchased item information.
Server receives, as input, the structured purchased item list. Server checks each product identifier against existing inventory records for the user, increments quantities for already registered items, and creates new inventory entries for newly introduced items. Server also records or updates expected use-by dates based on product catalog information and the purchase date. As output, server generates revised inventory records that incorporate transaction-derived quantities and stores them in the inventory table alongside sensor-derived and vision-derived records.

### Step 12

Server consolidates inventory records into a unified inventory snapshot.
Server takes, as input, multiple inventory sources, including entries derived from sensor data, visual detections, and transaction records. Server applies reconciliation rules to resolve discrepancies, for example preferring data from high-confidence sources or averaging estimates from multiple sources. Server sets a final quantity and state for each item, updates confidence scores, and stores a snapshot of the consolidated inventory in an inventory snapshot table. As output, server produces a coherent inventory state that can be used directly for prompt construction and proposal generation.

### Step 13

User inputs use conditions and a prompt sentence through the terminal.
User interacts with the terminal to select or enter conditions such as dietary preferences, health-related restrictions, number of servings, budget level, and maximum cooking time. User also inputs a free-form prompt sentence such as “Suggest a high-protein dinner for two people using what I have in my fridge.” Terminal collects, as input, the user's selections and text entries, encodes them into a structured request format, and attaches user identification. As output, terminal generates and sends a request containing use conditions and the prompt sentence to the server.

### Step 14

Server stores and structures use conditions and the prompt sentence.
Server receives, as input, the use conditions and prompt sentence from the terminal. Server writes the use conditions into a user profile or session table using explicit fields such as diet type, excluded ingredients, time limit, and serving count. Server also stores the raw prompt sentence and associates it with the current session and user identifier. As output, server produces structured profile records and a stored prompt string that can be retrieved and used by the prompt construction module.

### Step 15

Server constructs an extended prompt sentence for the generative AI model.
Server reads, as input, the consolidated inventory snapshot, the structured use conditions, and the stored prompt sentence. Server enumerates items from the inventory snapshot, filters items based on state (for example, prioritizing items near their use-by date), and formats them into a textual inventory list. Server appends explicit user conditions, such as “high-protein,” “low-fat,” and “two servings,” and then embeds the original user prompt sentence as the final instruction. Server concatenates these components in a predetermined template to generate a structured extended prompt sentence. As output, server produces the extended prompt sentence for use as input to the generative AI model.

### Step 16

Server invokes the generative AI model using the extended prompt sentence.
Server takes, as input, the extended prompt sentence and model control parameters such as temperature, maximum token length, and sampling strategy. Server tokenizes the prompt sentence into a sequence of tokens and sends the tokens to the generative AI model via a local inference engine or a remote API. The generative AI model processes the token sequence through internal layers and returns a generated token sequence that represents proposal information. As output, server receives a generated text string containing one or more proposals, such as recipes or meal plans that reference items from the inventory.

### Step 17

Server parses the generated proposal information into structured form.
Server accepts, as input, the generated text string from the generative AI model. Server splits the text into sections by headings or delimiters, identifies recipe titles, ingredient lists, and step-by-step instructions using pattern matching and parsing rules, and normalizes ingredient names and units. Server structures the parsed data into a proposal data model, where each proposal has associated ingredients, quantities, and instructions. As output, server produces a structured proposal dataset that can be compared with the inventory.

### Step 18

Server compares required items in proposals with inventory information.
Server reads, as input, the structured proposal dataset and the consolidated inventory snapshot. For each ingredient in each proposal, server normalizes the ingredient name, finds matching inventory entries using the product catalog and name normalization rules, and converts required units into base units if necessary. Server subtracts available quantities from required quantities to determine sufficiency or insufficiency for each ingredient. As output, server generates a comparison result that marks each ingredient as sufficient, partially sufficient, or insufficient.

### Step 19

Server identifies insufficient items and substitutable items.
Server takes, as input, the comparison result indicating which ingredients are not fully available. Server looks up substitution rules in a substitution table, where each rule associates an ingredient with one or more possible substitutes and conditions for substitution. Server checks the inventory snapshot for availability of substitute items and determines whether substitutions can satisfy the required quantities. Server then compiles a shortage list for truly missing items and a substitution list for ingredients that can be satisfied by available substitutes. As output, server generates structured shortage and substitution data linked to each proposal.

### Step 20

Server generates output data for presentation to the user.
Server accepts, as input, the structured proposal dataset, the shortage list, and the substitution list. Server packages these into an output structure that includes, for each proposal, a description, ingredient list, steps, and annotations about missing and substitutable items. Server may also include metadata such as estimated preparation time and nutritional characteristics if available. As output, server produces an output data message ready to be transmitted to the terminal.

### Step 21

Terminal receives and presents the output data to the user.
Terminal receives, as input, the output data message from the server. Terminal parses the message, organizes proposals into display pages or cards, and highlights missing ingredients and suggested substitutes with visual markers. Terminal renders recipe titles, ingredient lists, preparation steps, and messages such as “You are missing: 1 lemon (substitute available: 1 lime).” As output, terminal displays the proposals and associated shortage information on the screen so that the user can understand and act upon the recommendations.

### Step 22

User reviews proposals and optionally provides feedback.
User inspects, as input, the displayed proposals and associated shortage and substitution information. User may select a proposal to follow, adjust serving numbers, or reject certain ingredients. User may also confirm or override suggested substitutions. Terminal collects these interactions as feedback data and sends them back to the server. As output, user actions result in updated preferences or session records that the server can use to refine future prompt construction and proposal generation.

### Application Example 2

Description follows regarding a flow of the specific processing in an Application Example 2. The units of the system described below are implemented by the data processing device 12 and the smart device 14. The data processing device 12 is called a “server” and the smart device 14 is called a “terminal”.

Conventional computer-implemented menu recommendation systems primarily focus on rule-based selection from pre-defined recipe databases or simple filtering using user-specified conditions. Such systems typically do not integrate heterogeneous data sources, such as dynamically recognized inventory from sensors and images, optical character recognition results from receipts, and real-time user emotion signals, into a unified mechanism for generating prompts to a generative AI model. As a result, known systems exhibit several technical shortcomings.

First, with respect to data acquisition, conventional systems treat ingredient availability as static or manually entered information, which leads to incomplete or outdated inventory data. Image data from cameras, measurement data from sensors, and text data from receipts are not processed in a coordinated way at the computing architecture level, resulting in inefficient use of storage, increased manual input, and low reliability of the data pipeline.

Second, in terms of computational processing, existing systems do not provide a structured method for transforming multiple types of contextual data—such as inventory information, user meal conditions, user preferences, and emotion information—into machine-consumable prompt sentences for a generative AI model. Prompt construction is often ad hoc, manually tuned, or fixed, which prevents the computing system from flexibly exploiting the expressive power of generative AI models. This leads to poor personalization, suboptimal recommendations, and unnecessary iterative user interaction, thereby increasing processing overhead on both client and server.

Third, current approaches do not systematically leverage expiration information and storage state information to control menu generation at the algorithmic level. In particular, computing resources are not used to automatically prioritize items approaching expiration and to bias the generative AI model toward energy-efficient cooking plans. This causes redundant processing (e.g., repeated generation of menus that ignore near-expiry items), data inconsistencies in inventory management, and an inability to algorithmically reduce food waste and energy consumption.

Fourth, conventional notification subsystems generally output static or template-based messages, without adjusting message style or tone based on an automatically inferred emotional state of the user. From a computer-technical perspective, this means that emotion information, even if available, is not integrated as a parameter into the text generation pipeline, and the messaging subsystem cannot exploit generative capabilities to adapt notifications dynamically. This reduces the effectiveness of user interaction, may trigger additional unnecessary interactions, and wastes communication bandwidth and processing cycles.

Accordingly, there is a need for an improved computer-implemented system that (i) acquires and unifies multi-modal inventory and user context data, (ii) algorithmically generates structured prompt sentences for a generative AI model based on such unified data, (iii) guides the generative AI model to produce menus and meal plans that account for health conditions, inventory status, and resource optimization, and (iv) automatically generates emotion-adaptive notifications. Such a system should improve the overall computing workflow for menu generation, inventory management, and user communication, thereby enhancing processing efficiency, reducing redundant computation, and improving the quality and relevance of AI-generated outputs.

The specific processing by the specific processing unit 290 of the data processing device 12 in Application Example 2 is realized by the following means.

The present invention provides a server comprising a processor configured to acquire, via an information input/output device, meal condition information and preference information from a user; to acquire article information in a storage apparatus from a detection device including an imaging device and a measuring device, and to execute image recognition processing and quantity estimation processing on the article information to generate inventory information representing a list of available articles; to extract additional purchase article information from a voucher image by character recognition processing and integrate the additional purchase article information into the inventory information; to acquire emotion information indicating an emotional state of the user from an emotion estimation processing device and store the emotion information as user context; to generate, based on the inventory information, the meal condition information, the preference information, and the emotion information, a prompt sentence according to a template and to transmit the prompt sentence to a generative artificial intelligence model so as to instruct the generative artificial intelligence model to generate a menu or a meal plan; to analyze menu information obtained from the generative artificial intelligence model, to extract required article information from the menu information, and to specify shortage article information by comparing the required article information with the inventory information; to present the menu information and the shortage article information to the user and, based on the shortage article information, to generate and transmit provision request data for articles via an external service interface; and to perform message generation processing that changes a writing style or an expression tone of a notification sentence regarding the shortage article information and the menu information in accordance with the emotion information. This enables integrated acquisition and processing of heterogeneous contextual data, structured generation of prompt sentences for a generative AI model, automatic production of inventory-and health-aware menus and meal plans, and emotion-adaptive notification output, thereby improving the efficiency, reliability, and personalization of computer-implemented menu recommendation and inventory management.

The term “processor” refers to a hardware computation unit or a set of hardware computation units, such as one or more central processing units or one or more microcontrollers, that execute machine-readable instructions to perform data processing operations described in the present disclosure.

The term “information input/output device” refers to a hardware and software combination, such as a user terminal, display, input interface, or communication interface, that enables a user to input information to, and receive information from, the processor over a communication link.

The term “meal condition information” refers to data representing user-specified requirements or constraints for a meal, including but not limited to nutritional preferences, calorie limits, preparation time, dietary restrictions, cuisine types, or meal categories.

The term “preference information” refers to data indicating user-specific likes, dislikes, past selections, or ratings related to meals, ingredients, or recipes, which the processor uses to personalize generated menus or meal plans.

The term “storage apparatus” refers to an article storage device, such as a refrigerator, pantry, cabinet, or other container, that is configured to store food items or other articles and that can be monitored by sensors or imaging devices to obtain article information.

The term “detection device” refers to a device or a combination of devices, including at least an imaging device and a measuring device, that is configured to sense physical characteristics of articles in the storage apparatus and to output corresponding digital data.

The term “imaging device” refers to an optical sensing component, such as a camera or image sensor, configured to capture image data of a region inside or associated with the storage apparatus for subsequent image recognition processing.

The term “measuring device” refers to a sensing component, such as a weight sensor, quantity sensor, or identification reader, configured to measure or detect at least one physical parameter of an article, including but not limited to mass, count, volume, or identity.

The term “article information” refers to raw or processed data obtained from the detection device, including image data, measurement data, or identification data, that represents the presence, type, and quantity of articles in the storage apparatus.

The term “image recognition processing” refers to a computational procedure in which the processor analyzes image data, using pattern recognition or machine learning techniques, to identify or classify articles depicted in the image data.

The term “quantity estimation processing” refers to a computational procedure in which the processor determines or estimates quantities of articles, based on measurement data, image recognition results, or both, to produce numerical values representing available amounts.

The term “list of available articles” refers to structured data that enumerates articles determined to be present in the storage apparatus, each associated with at least an identifier and a quantity.

The term “inventory information” refers to data representing a current state of available articles, including types, quantities, and optionally expiration information or storage state information, maintained and updated by the processor.

The term “voucher image” refers to digital image data representing a document, such as a receipt, invoice, or purchase record, that includes textual information describing one or more purchased articles.

The term “character recognition processing” refers to an optical character recognition procedure in which the processor converts textual regions of a voucher image into machine-readable text data representing article names, quantities, or other purchase details.

The term “additional purchase article information” refers to data, extracted from a voucher image, indicating articles that have been newly purchased, including article identifiers and quantities, and intended to be reflected in the inventory information.

The term “emotion estimation processing device” refers to a computing component or service that analyzes user-related data, such as facial images, voice signals, or textual content, to derive emotion information indicating an emotional state of the user.

The term “emotion information” refers to data representing an inferred emotional state of the user, such as joy, sadness, anger, or fatigue, optionally accompanied by confidence values or related attributes.

The term “user context” refers to a set of data associated with a user, including at least meal condition information, preference information, inventory information related to that user, and emotion information, which the processor uses in decision-making and content generation.

The term “template” refers to a structured representation or pattern defining an arrangement of textual segments and variable fields, which the processor uses as a basis to generate a prompt sentence by inserting context-dependent data values.

The term “prompt sentence” refers to a machine-generated natural-language or semi-structured text that encodes contextual information, constraints, and instructions, and that is provided as input to a generative artificial intelligence model to control its output.

The term “generative artificial intelligence model” refers to a trained computational model, such as a neural network configured for language generation, that receives a prompt sentence and outputs generated content including menus, meal plans, or notification text.

The term “menu” refers to a set of one or more proposed dishes or recipes, each optionally including a name, required articles, and preparation instructions, suitable for consumption in a meal.

The term “meal plan” refers to a structured arrangement of one or more menus over a specified time frame, such as a day or a week, optionally including scheduling, nutritional balance, and resource usage considerations.

The term “menu information” refers to data output by the generative artificial intelligence model that describes at least one menu or meal plan, including dish names, required articles, and optionally preparation steps and ancillary attributes.

The term “required article information” refers to data derived from menu information that specifies articles and associated quantities needed to realize a proposed menu or meal plan.

The term “shortage article information” refers to data identifying articles for which required quantities, as indicated by required article information, exceed corresponding quantities in the inventory information.

The term “external service interface” refers to a communication interface, such as an application programming interface, that enables the processor to interact with an external service, including but not limited to an article delivery service or ordering system.

The term “provision request data” refers to data generated by the processor for transmission to an external service, the data specifying articles, quantities, and optionally delivery or provisioning parameters, for procuring shortage articles.

The term “notification sentence” refers to a text message generated by the processor for presentation to the user, the message including at least information regarding menu information, shortage article information, or ordering status.

The term “writing style” refers to linguistic characteristics of a notification sentence, including but not limited to formality, politeness level, and sentence structure, which can be modified by the processor.

The term “expression tone” refers to an affective or emotional nuance conveyed by a notification sentence, such as calm, neutral, or enthusiastic, which the processor adjusts in accordance with emotion information.

The term “nutrition condition” refers to constraints or preferences related to nutritional properties of meals, such as calorie limits, macronutrient ratios, or inclusion or exclusion of specific nutrient categories.

The term “intake amount condition” refers to constraints on amounts of food or nutrients to be consumed, including per-meal or per-day quantity limits or targets specified by or for the user.

The term “expiration information” refers to data indicating a temporal limit of recommended use for an article, such as an expiration date, best-before date, or remaining days until expiration.

The term “storage state information” refers to data indicating physical or environmental conditions of articles in the storage apparatus, such as temperature, location, or open/closed status of containers, which may influence usability of the articles.

The term “cooking timing” refers to temporal parameters associated with preparation of meals, including but not limited to recommended preparation times, scheduling constraints, or priority levels for using particular articles.

In one embodiment, a server, a terminal, and one or more storage apparatuses cooperate to implement the claimed system. The server includes at least one processor, a main memory, a non-volatile storage device, and a network interface. The terminal includes an input/output interface, a display, at least one input device, and optionally a camera and a microphone. The storage apparatus, such as a refrigerator or pantry, includes a detection device having an imaging device and a measuring device. These components are interconnected via a communication network.

The server executes an operating system and one or more application programs implemented, for example, using a general-purpose programming language. The server further executes specialized libraries for image recognition, character recognition, and machine learning, such as an image processing framework, an optical character recognition engine, and a machine learning framework. The generative AI model is provided as a neural network-based language generator accessible via an application programming interface. The terminal executes a client application, such as a native mobile application or a web browser, which communicates with the server via a secure transport protocol.

The server acquires meal condition information and preference information from the user via the terminal. The terminal displays, on its display, a user interface including selectable controls for specifying nutritional preferences, calorie limits, desired preparation time, dietary restrictions, and cuisine types. The user operates the terminal by touch or pointing device and enters text such as “healthy dinner using chicken and broccoli, under 500 kcal” or “quick pasta recipe for two persons.” The terminal converts these user inputs into structured data, such as key-value pairs representing conditions and preferences, and transmits the structured data to the server via the network interface.

The server receives the structured data and stores it in the memory and in a user context data structure in the non-volatile storage. The server may represent the meal condition information and preference information as records in a relational table or as documents in a document-oriented database, each record associated with a user identifier. By storing the information in a structured form, the server can later combine it with inventory information and emotion information. This structured representation improves data management and enables efficient retrieval and updating of user-related context, thereby improving processing speed and reducing memory fragmentation.

The storage apparatus includes an imaging device such as a digital camera and a measuring device such as a weight sensor, an identification reader, or another quantity-detecting sensor.

The imaging device captures images of an internal region of the storage apparatus where articles are stored. The measuring device outputs analog or digital signals representing weight, count, or presence of articles placed on or near the sensor. A microcontroller in the storage apparatus or an associated gateway device converts these signals into digital data and transmits both image data and measurement data to the server, either directly or via the terminal.

The server executes image recognition processing on the image data using a convolutional neural network. In one example, the server loads a trained convolutional neural network comprising multiple convolutional layers, pooling layers, non-linear activation layers, and fully connected classification layers. Input images are resized to a fixed resolution and normalized. The server feeds the processed images into the network and obtains class probability vectors for each detected object region. The server then applies non-maximum suppression and thresholding rules to determine which classes are present, thereby identifying article types such as “chicken,” “tomato,” or “milk.” The server correlates these identified article types with measurement values from the measuring device, such as weight or count, to estimate quantities of articles. The server stores this information in an inventory data structure, such as a table with fields for article identifier, quantity, expiration information, and storage state information.

The server improves computational efficiency by performing batch processing of image data and by using vectorized operations for convolution and pooling. The server may also employ model compression techniques, such as weight quantization or pruning, to reduce the computational cost of the convolutional neural network. As a result, the server can process multiple images quickly and update inventory information in near real time, reducing latency between physical changes in the storage apparatus and their reflection in the digital inventory.

The terminal or the storage apparatus acquires voucher images representing receipts or purchase records by using a camera. The terminal transmits the voucher images to the server.

The server executes character recognition processing by applying an optical character recognition algorithm. The server segments the voucher image into text regions, binarizes the regions, and recognizes characters using a trained character recognition model. The server then parses lines of recognized text to identify article names, quantities, and prices. For example, the server may detect text lines such as “CHICKEN BREAST 500 g” or “BROCCOLI 1.” The server maps these recognized names onto internal article identifiers by using a mapping table or a classification model, and then updates the inventory data structure by adding or updating article records. This automated integration of receipt-derived data reduces manual input and synchronizes the inventory information with actual purchases, thereby increasing accuracy and reducing errors compared to manual inventory tracking.

The server further acquires emotion information from an emotion estimation processing device. In one implementation, the terminal captures facial images and voice signals of the user by using its camera and microphone. The terminal transmits these signals, or features derived from them, to the server or to a separate emotion estimation processing device. The emotion estimation device may implement a neural network including convolutional layers for facial feature extraction and recurrent or attention-based layers for temporal aggregation, or may implement a spectrogram-based model for voice analysis. The device outputs emotion labels such as “joy,” “sadness,” “anger,” or “tiredness,” together with confidence values. The server receives the emotion information and stores it as part of the user context. The server may aggregate emotion information over time to compute trends or average emotional states, thereby providing richer context for subsequent processing.

The server generates a prompt sentence to be provided as input to the generative AI model. To do so, the server reads the current inventory information, the meal condition information, the preference information, and the emotion information from the data structures in memory.

The server then applies a template-based prompt generation procedure. The server maintains a template that defines a sequence of segments, such as an available-articles segment, a condition segment, an emotion segment, and an instruction segment. The server inserts concrete values into placeholders in the template to construct a complete prompt sentence.

For example, the server may generate the following prompt sentence:
“Available ingredients: chicken 200 g, broccoli 1 head, tomato 2, olive oil, salt, pepper. User conditions: healthy dinner, under 500 kcal, easy to cook. User emotion: tired. Please propose one comforting and simple dinner recipe using mainly these ingredients. Output the recipe name, ingredient list with quantities, approximate calories, and concise cooking steps.”

In another example, the server may generate:
“There are tomatoes and mozzarella cheese in the refrigerator. The user wants a healthy low-calorie dinner. The user's emotion is joy. Please propose a celebratory but healthy menu using these ingredients. Output the menu name, required ingredients, and step-by-step cooking instructions.”

By constructing prompt sentences in a structured, template-based manner, the server ensures that heterogeneous contextual data are encoded in a consistent, unambiguous form that the generative AI model can interpret effectively. This improves the relevance and coherence of the generated menus and meal plans. Furthermore, the template-based approach allows the server to modify or extend the prompt structure systematically, which improves maintainability and enables optimization of prompt design based on empirical performance data.

The generative AI model is implemented as a transformer-based neural network including an embedding layer, multiple self-attention layers, feedforward layers, and an output projection layer. The model is trained on large-scale textual data including recipe descriptions, ingredient lists, and cooking instructions. During training, the model minimizes a loss function such as a cross-entropy loss between predicted token distributions and ground-truth tokens. The model parameters, including weights in attention and feedforward layers, are updated by gradient-based optimization, such as stochastic gradient descent or an adaptive optimizer, based on mini-batches of training examples. In some embodiments, the model is further fine-tuned on domain-specific data related to cooking and menus.

The server transmits the prompt sentence to the generative AI model via an application programming interface. The server passes the prompt sentence as input tokens, along with configuration parameters such as generation length and diversity settings. The generative AI model computes contextual embeddings for each token and applies self-attention to capture relationships among tokens in the prompt. The output layer produces probability distributions for subsequent tokens, and the model sequentially generates text representing menus, recipes, or meal plans. The generated text may include recipe names, required ingredients with quantities, cooking times, and detailed preparation steps. Because the model is conditioned on specific inventory, conditions, and emotion data encoded in the prompt sentence, the resulting output is tailored to the current context and user state.

The server receives the generated text and parses it into structured menu information. The server may instruct the generative AI model to output the results in a partially structured text format, such as labeled sections “Recipe Name:”, “Ingredients:”, and “Steps:”. The server scans for these labels and segments the text accordingly. For each recipe, the server extracts the required article information, including article names and quantities. The server normalizes article names by mapping them to internal identifiers used in the inventory data structure, which may involve simple string matching, synonym tables, or a lightweight classifier. This normalization enables direct comparison between required article quantities and available inventory quantities.

The server compares the required article information with the inventory information to determine shortage article information. For each article required by a recipe, the server subtracts the available quantity in the inventory from the required quantity. If the result is negative or insufficient, the server records the difference as a shortage. The server aggregates all such differences into a shortage article list associated with the corresponding recipe.

Because this computation is performed in the server's memory using structured numeric data, it can be executed efficiently even for multiple recipes and large inventories. This systematic comparison reduces errors that might occur if a user were to manually inspect inventory and determine shortages.

To generate messages for the user, the server creates notification sentences describing the menu information and shortage article information. The server incorporates emotion information in order to adjust the writing style and expression tone. In one embodiment, the server chooses among several predefined tone styles (for example, calm, neutral, and cheerful) based on the emotion label. The server then constructs notification sentences by applying different lexical choices and syntactic structures depending on the tone. In another embodiment, the server transmits a short prompt sentence to the generative AI model to generate tone-adapted notifications, such as:
“User emotion: anger. Generate a calm and gentle notification that says ‘basil is missing for the Caprese recipe’.”

By performing this tone adaptation at the server side based on emotion information, the system provides more effective communication and may reduce negative reactions or confusion. From a technical standpoint, this integration of emotion as a parameter in the text generation pipeline represents a non-conventional use of emotion analysis results and leads to dynamically customized messages without requiring multiple static templates.

The terminal receives the menu information and shortage article information from the server in structured form. The terminal displays recipes, ingredients, and missing items to the user on the display. Articles that are available are indicated differently from articles that are missing. The user may select a particular menu or recipe for execution. The terminal also provides controls for ordering missing articles from an external service. When the user initiates a provision request, the terminal sends a corresponding request to the server.

The server then generates provision request data by mapping shortage article information to product codes or service-specific item identifiers used by an external service. The server constructs a request including item identifiers, quantities, and delivery parameters, and transmits the request via the external service interface. The external service responds with confirmation information, such as an order identifier and estimated delivery time. The server stores this information and sends a summary back to the terminal. Thus, the system connects analysis of digital data (inventory and menus) with control and utilization of real-world services, resulting in physical delivery of required articles to the user.

The described configuration yields several technical effects beyond mere automation of human tasks. By unifying image-based inventory recognition, receipt-based updates, and user context into a single data pipeline, the server reduces inconsistencies between actual physical stock and stored digital records. This reduces the need for repeated network calls and human corrections and improves the reliability of subsequent computations. The template-driven prompt generation allows the server to encode multiple context dimensions into compact prompt sentences, improving efficiency in communication with the generative AI model. By structuring inputs and outputs of the generative AI model, the server can perform direct numeric comparisons and manipulations on data, enabling faster and more accurate computation of shortages and recommendations.

Furthermore, the use of expiration information and storage state information in the prompt sentence aligns the output of the generative AI model with resource optimization goals. For example, the server may generate a prompt sentence such as:
“Available ingredients include chicken (expires in 1 day), broccoli (expires in 3 days), and rice (expires in 10 days). Please propose menus that prioritize using ingredients with the shortest remaining shelf life and minimize total cooking energy.”

This explicit encoding of expiration and energy-related conditions guides the generative AI model to propose menus that reduce food waste and energy consumption. By automating this logic in the server, the system can consistently apply such optimization strategies at scale, which would be difficult for a human user to do repeatedly and accurately.

The internal architecture of the server enhances computation by dividing responsibilities among modules: an acquisition module for receiving sensor and voucher data; a recognition module for performing image and character recognition; a context management module for storing and retrieving user and inventory context; a prompt generation module for constructing prompt sentences; a generative interaction module for communicating with the generative AI model; a parsing module for interpreting generated text; a shortage computation module; and a notification module for generating emotion-aware notification sentences. Each module operates on well-defined data structures and interfaces, allowing pipeline optimization and reuse. For example, the recognition module can batch process images to exploit hardware acceleration, while the prompt generation module can cache frequently used phrase segments to reduce string processing overhead.

Alternative embodiments may vary the type of sensors in the storage apparatus. For example, instead of or in addition to a weight sensor, a radio-frequency identification reader can be used to detect articles tagged with identification devices. The server, in this case, receives identification codes and uses them to index records in the inventory data structure. The measuring device can also be a volume sensor or a count sensor. The generative AI model can also be implemented locally on the server or distributed across multiple computing nodes. Different neural network architectures, such as recurrent neural networks or encoder-decoder architectures with attention, can be used instead of a transformer. In all such variations, the processor still performs the operations of acquiring multi-modal context, generating structured prompt sentences, invoking a generative AI model, computing shortages, and generating emotion-aware notifications.

In summary, the server, the terminal, and the storage apparatus cooperate to implement a system in which the processor executes specific technical operations on hardware-captured data streams and structured context data. The system improves computer technology by (i) structuring heterogeneous sensor, receipt, and user context data into coherent data structures for efficient processing, (ii) generating context-rich prompt sentences to control a generative AI model in a non-conventional, template-based manner, (iii) performing precise, automated comparison between generated requirements and inventory data to determine shortages, and (iv) integrating emotion information into message generation to adapt notification style at runtime. These features yield improved processing speed, higher accuracy of inventory and menu suggestions, reduced communication overhead with external services, and more effective user interaction compared to conventional systems that rely solely on static rule-based logic or manual processes.

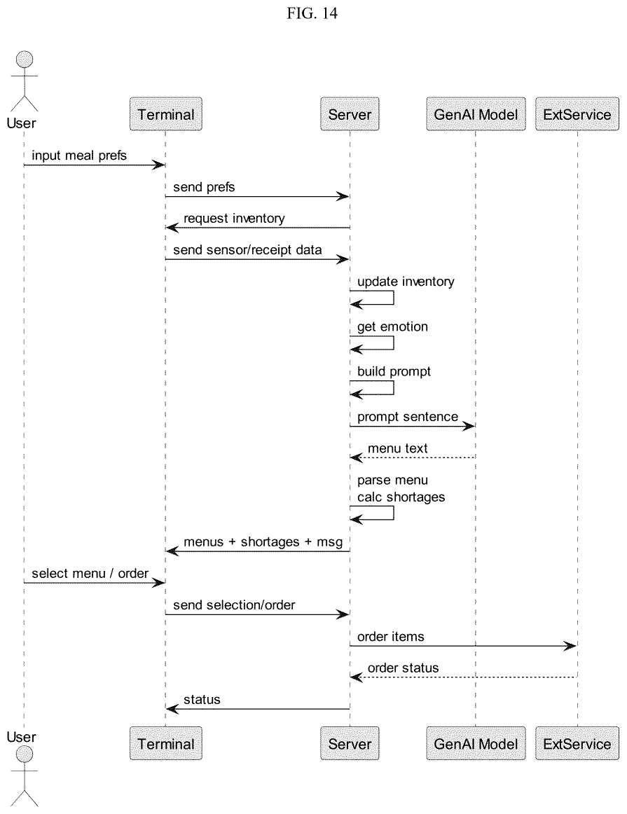

The following describes the processing flow using FIG. 14.

### Step 1

The user operates the terminal to input meal conditions and preferences.
The user selects options such as “healthy”, “low calorie”, “quick dinner”, and enters free text such as “use chicken and broccoli for dinner under 500 kcal”.
Input: raw user interactions (touch events, key presses, speech-to-text results).
The terminal converts these interactions into structured condition data (for example, key-value pairs like {type: “dinner”, max_cal: 500, main_ingredients: [“chicken”, “broccoli”]}).
Output: structured meal condition information and preference information, stored in the terminal's memory.

### Step 2

The terminal transmits the structured meal condition information and preference information to the server.
Input: structured condition data and a user identifier.
The terminal serializes the data into a request payload and sends it over a secure network protocol (for example, HTTPS) to an API endpoint on the server.
Output: a network request containing meal condition information and preference information, delivered to the server.

### Step 3

The server receives and stores the meal condition information and preference information.
Input: the request payload from the terminal.
The server parses the payload, validates fields, and writes the data into a user context data structure in persistent storage (for example, relational tables or document records).
The server may normalize units (for example, minutes, calories) and categorical values (for example, cuisine types) during this write operation.
Output: normalized and stored meal condition information and preference information associated with the user.

### Step 4

The storage apparatus captures physical inventory data using the imaging device and measuring device.
Input: current physical state of articles in the storage apparatus.
The imaging device acquires images of the interior, and the measuring device (for example, weight sensor, identification reader) outputs signals corresponding to articles present.
The storage apparatus or a gateway microcontroller digitizes these signals and transmits image data and measurement data to the server, optionally via the terminal.
Output: digitized article information including image data and measurement data, received by the server.

### Step 5

The server performs image recognition and quantity estimation to create inventory information.
Input: article information (image data and measurement data).
The server applies a convolutional neural network to the image data to classify objects into article categories, and combines classification results with measurement values to estimate quantities of each article.
The server executes data processing such as resizing and normalizing images, running forward passes through the neural network, applying thresholds, and mapping detected classes to article identifiers.
Output: an inventory list containing article identifiers, estimated quantities, and optionally initial expiration and storage state information, stored as inventory information.

### Step 6

The terminal captures voucher images representing recent purchases.
Input: a physical receipt or purchase document.
The user uses the terminal's camera to scan the receipt; the terminal acquires the voucher image and compresses it if necessary.
Output: a voucher image file transmitted from the terminal to the server.

### Step 7

The server executes character recognition on the voucher image and updates the inventory.
Input: voucher image.
The server runs an optical character recognition algorithm to detect text regions, recognize characters, and extract lines of text describing purchased articles and quantities.
The server parses the recognized text, maps each product name to an internal article identifier, and increments or adds corresponding entries in the inventory information.
Output: updated inventory information including additional purchase article information.

### Step 8

The terminal or a separate emotion estimation device acquires emotion-related data from the user.
Input: user facial images, voice signals, and optionally text input.
The terminal captures these data using its camera and microphone and transmits them, or extracted features, to the server or to an emotion estimation device.
Output: raw or preprocessed emotion-related data ready for emotion estimation.

### Step 9

The server acquires emotion information from the emotion estimation processing device.
Input: emotion-related data or processed emotion analysis results.
The emotion estimation device applies a trained model (for example, neural networks for facial or voice emotion recognition) and outputs an emotion label and confidence scores.
The server receives this emotion information, associates it with the user context, and writes it into a user context record.
Output: stored emotion information (for example, “tired”, “joy”) linked to the user.

### Step 10

The server aggregates context data required for prompt sentence generation.
Input: meal condition information, preference information, inventory information, and emotion information from the database.
The server queries and combines these records into an internal context object, normalizing formats (for example, lists of ingredients, numerical limits, current emotion).
The server filters out expired articles and optionally ranks articles by remaining shelf life and usage priority.
Output: a consolidated context object containing cleaned and normalized data for prompt generation.

### Step 11

The server generates a prompt sentence for the generative AI model based on the consolidated context.
Input: consolidated context object (available articles, user conditions, preferences, emotion).
The server applies a template defining segments such as “Available ingredients”, “User conditions”, “User emotion”, and “Instructions for output”.
The server fills placeholders with concrete values, concatenates segments into a coherent natural-language text, and may add constraints such as calorie limits and time limits.
For example, the server generates the prompt sentence:
“Available ingredients: chicken 200 g, broccoli 1 head, tomato 2, olive oil, salt, pepper. User conditions: healthy dinner, under 500 kcal, easy to cook. User emotion: tired. Please propose one comforting and simple dinner recipe using mainly these ingredients. Output the recipe name, ingredient list with quantities, approximate calories, and concise cooking steps.”
Output: a complete prompt sentence stored as a text string in memory.

### Step 12

The server transmits the prompt sentence to the generative AI model and obtains generated menu information.
Input: prompt sentence and generation parameters (for example, maximum tokens, temperature).
The server sends a request to the generative AI model's interface, including the prompt sentence and parameters, and the generative AI model processes the text using its neural network to generate continuation text.
The server receives the generated text describing menus, recipes, or meal plans.
Output: raw generated menu text returned by the generative AI model.

### Step 13

The server parses the generated menu text into structured menu information.
Input: raw generated menu text.
The server identifies sections such as recipe names, ingredient lists, and preparation steps by scanning for markers (for example, “Recipe Name:”, “Ingredients:”, “Steps:”) or using text parsing rules.
The server splits the text into separate recipe records, extracts required article names and quantities, and stores them as structured data (for example, arrays or records).
Output: structured menu information for one or more recipes, including required article information and optional attributes (for example, cooking time, calories).

### Step 14

The server computes shortage article information by comparing required article information with inventory information.
Input: structured menu information and current inventory information.
The server, for each recipe, iterates through required articles, retrieves corresponding available quantities from inventory records, and subtracts available quantities from required quantities.
The server records any article for which the required quantity exceeds the available quantity, with the missing amount, as shortage article information.
Output: a shortage article list linked to each recipe.

### Step 15

The server generates notification sentences for menus and shortages with tone adaptation based on emotion information.
Input: menu information, shortage article information, and emotion information.
The server selects a tone (for example, calm, neutral, cheerful) according to the emotion label and constructs notification sentences using tone-specific wording and sentence structure.
Alternatively, the server generates a short prompt sentence such as “User emotion: anger. Generate a calm and gentle notification that says ‘basil is missing for the Caprese recipe’.” and transmits it to the generative AI model to obtain an emotion-adapted notification text.
Output: one or more notification sentences describing menus and shortages with an adapted writing style and expression tone.

### Step 16

The server transmits the menu information, shortage article information, and notification sentences to the terminal.
Input: structured menu information, shortage article list, and notification sentences.
The server packs these data into a response payload, including identifiers, names, ingredient lists, and text messages, and sends the payload via the network interface to the terminal.
Output: a response message containing all necessary presentation data delivered to the terminal.

### Step 17

The terminal displays the menus, shortages, and notifications to the user. Input: response payload from the server.
The terminal parses the payload and renders user interface elements showing recipe titles, ingredients grouped into “available” and “missing”, and the notification sentences in an appropriate area of the screen.
The terminal may highlight near-expiration articles or indicate health-related attributes, based on data fields in the payload.
Output: a visual presentation on the terminal display, enabling the user to understand the proposed menus and shortages.

### Step 18

The user reviews the proposed menus and selects a desired menu or menus.
Input: visual presentation of menus and shortages.
The user interacts with the terminal by selecting a recipe, marking it as chosen, and optionally adjusting the number of servings or excluding certain ingredients.
The terminal records the user's selection and feedback as interaction data.
Output: selected menu identifiers and feedback data stored in the terminal's memory.

### Step 19

The terminal transmits user selections and feedback to the server.
Input: selected menu identifiers and feedback (for example, ratings, acceptance flags).
The terminal packages this data into a request and sends it to the server via the network.
Output: a feedback request received by the server.

### Step 20

The server updates user preference information based on feedback.
Input: feedback data and existing preference information.
The server updates statistics such as counts of accepted recipes by category, average ratings for ingredients, and rejection patterns, by incrementing counters or adjusting preference scores.
The server stores updated preference information in the user context data structure for use in future prompt generation and menu ranking.
Output: updated preference information that refines personalization over time.

### Step 21

The user initiates a provision request for missing articles through the terminal.
Input: displayed shortage article list.
The user uses the terminal to select missing articles to be ordered and confirms a provision request (for example, by tapping “Order missing items”).
The terminal collects the selected articles and user confirmation.
Output: a provision request set containing selected shortage articles.

### Step 22

The terminal sends the provision request set to the server.
Input: list of selected shortage article identifiers and quantities.
The terminal creates a request payload and transmits it to the server over the network.
Output: provision request data received at the server.

### Step 23

The server generates provision request data for an external service and transmits it via an external service interface.
Input: provision request set and mapping information between internal article identifiers and external product identifiers.
The server translates internal article identifiers to external service product codes, composes an order structure including product codes, quantities, delivery address, and possibly payment tokens, and sends the order structure through the external service interface.
Output: an order request transmitted to the external service and an internal record of the order.

### Step 24

The server receives confirmation from the external service and notifies the terminal.
Input: response data from the external service, including order identifier, status, and estimated delivery time.
The server stores the confirmation in an order repository and creates a summary message describing ordered items and delivery details.
The server transmits the summary message and updated order status to the terminal.
Output: an order confirmation payload sent to the terminal.

### Step 25

The terminal displays order status and related notifications to the user.
Input: order confirmation payload.
The terminal shows the order identifier, items ordered, estimated delivery time, and any additional messages to the user, and may provide actions such as “track order” or “cancel order”.
The terminal may also refresh the view of inventory-related information once the order is expected to be fulfilled.
Output: an updated user interface reflecting external provisioning actions and current order status.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Moreover, although the processing by the data processing system 10 described above was executed by the specific processing unit 290 of the data processing device 12 or by the control unit 46A of the smart device 14, the processing may be executed by a specific processing unit 290 of the data processing device 12 and a control unit 46A of the smart device 14. Moreover, the specific processing unit 290 of the data processing device 12 acquires and collects information needed for processing from the smart device 14 or from an external device or the like, and the smart device 14 acquires and collects information needed for processing from the data processing device 12 or from an external device or the like.

For example, a collection unit is implemented by the control unit 46A of the smart device 14 and/or by the specific processing unit 290 of the data processing device 12. For example, an acquisition unit acquires number-of-steps data using the camera 42 and/or the communication I/F 44 of the smart device 14, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the output device 40 of the smart device 14 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart device 14.

### Second Exemplary Embodiment

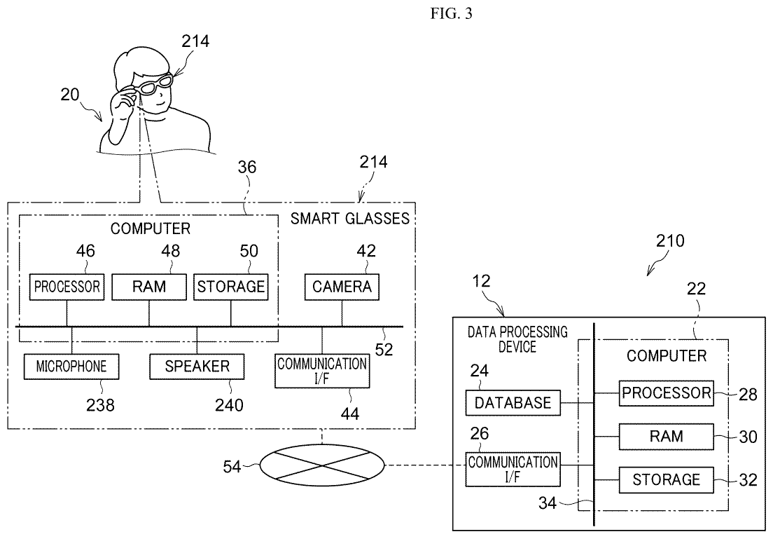

FIG. 3 illustrates an example of a configuration of a data processing system 210 according to a second exemplary embodiment.

As illustrated in FIG. 3, the data processing system 210 includes a data processing device 12 and smart glasses 214. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The smart glasses 214 include a computer 36, a microphone 238, a speaker 240, a camera 42, and a communication I/F 44. The computer 36 includes a processor 46, RAM 48, and storage 50. The processor 46, the RAM 48, and the storage 50 are connected to a bus 52. The microphone 238, the speaker 240, the camera 42, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

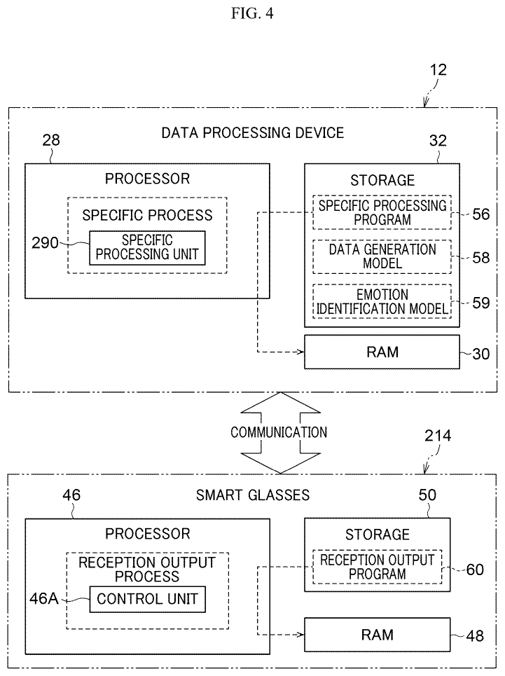

FIG. 4 illustrates an example of relevant functions of the data processing device 12 and the smart glasses 214. As illustrated in FIG. 4, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290. The specific processing unit 290 uses the emotion identification model 59 to estimate an emotion of a user, and is able to perform the specific processing using the user emotion. In an emotion estimation function (emotion identification function) that uses the emotion identification model 59, various estimations, predictions, and the like are performed related to emotions of the user, include estimating and predicting the emotion of the user, however, there is no limitation to such examples. Moreover, estimation and prediction of emotion also includes, for example, analyzing (parsing) emotions and the like.

Reception and output processing is performed by the processor 46 in the smart glasses 214. A reception and output program 60 is stored in the storage 50. The processor 46 reads the reception and output program 60 from the storage 50 and in the RAM 48 executes the read reception and output program 60. The reception and output processing is implemented by the processor 46 operating as the control unit 46A according to the reception and output program 60 executed in the RAM 48. Note that a configuration may be adopted in which the smart glasses 214 include a data generation model and an emotion identification model similar to the data generation model 58 and the emotion identification model 59, and processing similar to the specific processing unit 290 is performed using these models.

Next, description follows regarding the specific processing by the specific processing unit 290 of the data processing device 12. The units of the system described below are implemented by the data processing device 12 and the smart glasses 214. In the following description the data processing device 12 is called a “server”, and the smart glasses 214 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the smart glasses 214. The control unit 46A in the smart glasses 214 outputs the specific processing result to the speaker 240. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the smart glasses 214, the processing may be executed by a specific processing unit 290 of the data processing device 12 and a control unit 46A of the smart glasses 214. Moreover, the specific processing unit 290 of the data processing device 12 acquires and collects information needed for processing from the smart glasses 214 or from an external device or the like, and the smart glasses 214 acquires and collects information needed for processing from the data processing device 12 or from an external device or the like.

For example, the collection unit is implemented by the control unit 46A of the smart glasses 214 and/or by the specific processing unit 290 of the data processing device 12. For example, an acquisition unit acquires number-of-steps data using the camera 42 and/or the communication I/F 44 of the smart glasses 214, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 of the smart glasses 214 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the smart glasses 214.

### Third Exemplary Embodiment

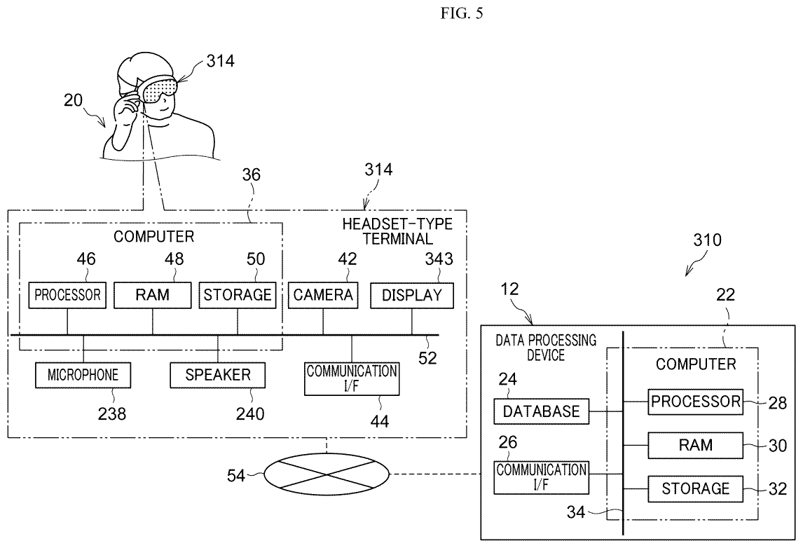

FIG. 5 illustrates an example of a configuration of a data processing system 310 according to a third exemplary embodiment.

As illustrated in FIG. 5, the data processing system 310 includes a data processing device 12 and a headset-type terminal 314. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The headset-type terminal 314 includes a computer 36, a microphone 238, a speaker 240, a camera 42, a communication I/F 44, and a display 343. The computer 36 includes a processor 46, RAM 48, and storage 50. The processor 46, the RAM 48, and the storage 50 are connected to a bus 52. The microphone 238, the speaker 240, the camera 42, the display 343, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the user 20 (for example, an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

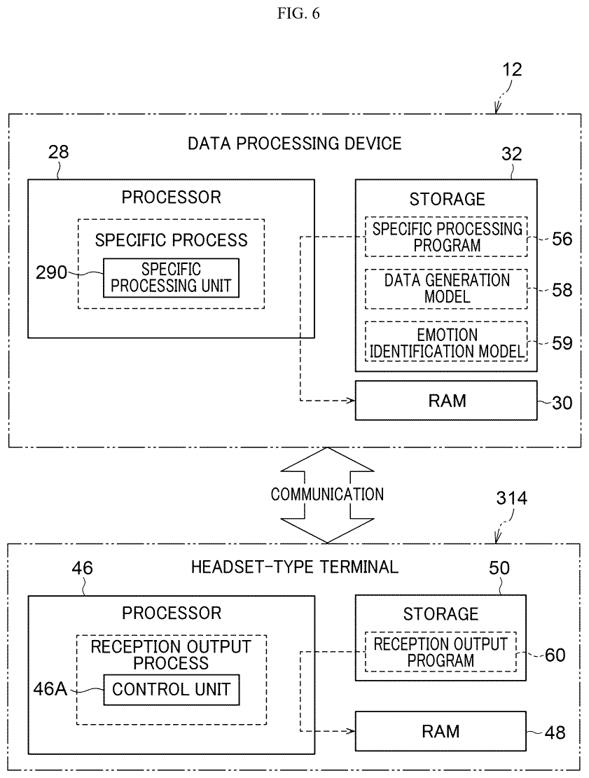

FIG. 6 illustrates an example of relevant functions of the data processing device 12 and the headset-type terminal 314. As illustrated in FIG. 6, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the headset-type terminal 314. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the headset-type terminal 314. In the following description the data processing device 12 is called a “server”, and the headset-type terminal 314 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the headset-type terminal 314. In the headset-type terminal 314, the control unit 46A outputs the result of the specific processing to the speaker 240 and the display 343. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the headset-type terminal 314, the processing may be executed by a specific processing unit 290 of the data processing device 12 and a control unit 46A of the headset-type terminal 314. Moreover, the specific processing unit 290 of the data processing device 12 acquires and collects information needed for processing from the headset-type terminal 314 or from an external device or the like, and the headset-type terminal 314 acquires and collects information needed for processing from the data processing device 12 or from an external device or the like.

For example, the collection unit is implemented by the control unit 46A of the headset-type terminal 314 and/or by the specific processing unit 290 of the data processing device 12. For example, an acquisition unit acquires number-of-steps data using the camera 42 and/or the communication I/F 44 of the headset-type terminal 314, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the display 343 of the headset-type terminal 314 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the headset-type terminal 314.

### Fourth Exemplary Embodiment

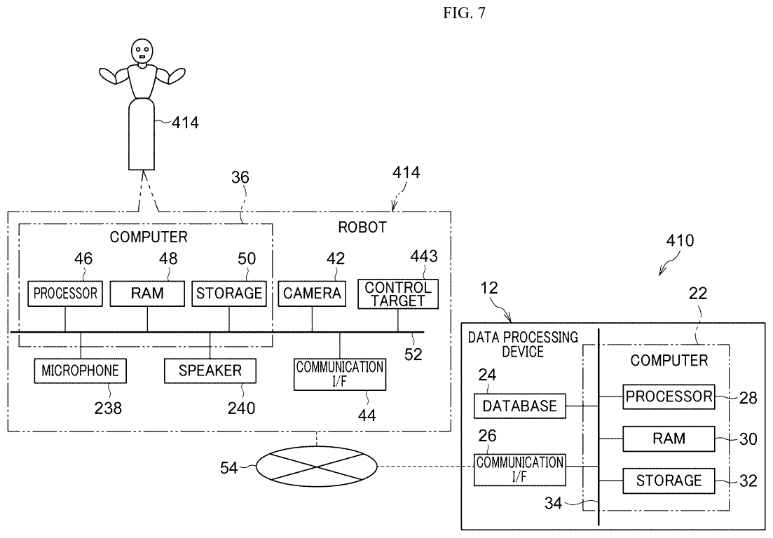

FIG. 7 illustrates an example of a configuration of a data processing system 410 according to a fourth exemplary embodiment

As illustrated in FIG. 7, the data processing system 410 includes a data processing device 12 and a robot 414. A server is an example of the data processing device 12.

The data processing device 12 includes a computer 22, a database 24, and a communication I/F 26. The computer 22 is an example of a “computer” according to technology disclosed herein. The computer 22 includes a processor 28, RAM 30, and storage 32. The processor 28, the RAM 30, and the storage 32 are connected to a bus 34. The database 24 and the communication I/F 26 are also connected to the bus 34. The communication I/F 26 is connected to a network 54. Examples of the network 54 include a Wide Area Network (WAN) and/or a local area network (LAN).

The robot 414 includes a computer 36, a microphone 238, a speaker 240, a camera 42, a communication I/F 44, and a control target 443. The computer 36 includes a processor 46, RAM 48, and storage 50. The processor 46, the RAM 48, and the storage 50 are connected to a bus 52. The microphone 238, the speaker 240, the camera 42, the control target 443, and the communication I/F 44 are also connected to the bus 52.

The microphone 238 receives an instruction or the like from a user 20 by receiving speech uttered by the user 20. The microphone 238 captures the speech uttered by the user 20, converts the captured speech into audio data, and outputs the audio data to the processor 46. The speaker 240 outputs audio under instruction from the processor 46.

The camera 42 is a compact digital camera installed with an optical system such as a lens, an aperture, a shutter, and the like, and with an imaging device such as a complementary metal-oxide semiconductor (CMOS) image sensor or a charge coupled device (CCD) image sensor or the like. The camera 42 images the surroundings of the robot 414 (for example, with an imaging range defined by an angle of view equivalent to the width of visual field of an ordinary healthy subject).

The communication I/F 44 is connected to the network 54. The communication I/F 44 and the communication I/F 26 perform the role of exchanging various information between the processor 46 and the processor 28 over the network 54. The exchange of various information between the processor 46 and the processor 28 is performed in a secure state using the communication I/F 44 and the communication I/F 26.

The control target 443 includes a display device, eye LEDs, and motors to drive arms, hands, feet, and the like. The posture and gesture of the robot 414 are controlled by controlling the motors of the arms, hands, feet, and the like. Part of an emotion of the robot 414 can be expressed by controlling these motors. Moreover, a facial expression of the robot 414 can be represented by controlling an illumination state of the eye LEDs of the robot 414.

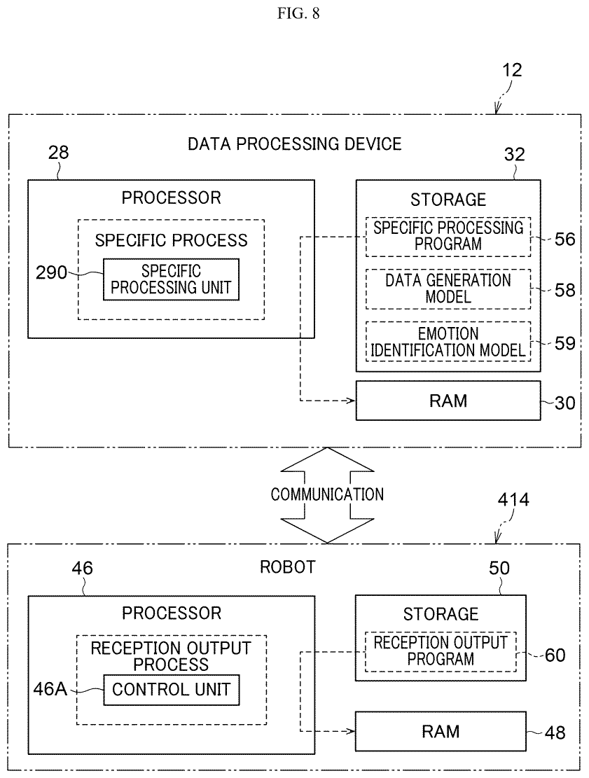

FIG. 8 illustrates an example of relevant functions of the data processing device 12 and the robot 414. As illustrated in FIG. 8, specific processing is performed by the processor 28 in the data processing device 12. A specific processing program 56 is stored in the storage 32.

The specific processing program 56 is an example of a “program” according to technology disclosed herein. The processor 28 reads the specific processing program 56 from the storage 32, and in the RAM 30 executes the read specific processing program 56. The specific processing is implemented by the processor 28 operating as the specific processing unit 290 according to the specific processing program 56 executed in the RAM 30.

The data generation model 58 and the emotion identification model 59 are stored in the storage 32. The data generation model 58 and the emotion identification model 59 are employed by the specific processing unit 290.

Reception and output processing is performed by the processor 46 in the robot 414. A reception and output program 60 is stored in the storage 50. The processor 46 reads the reception and output program 60 from the storage 50, and in the RAM 48 executes the read reception and output program 60. The reception and output processing is implemented by the processor 46 operating as the control unit 46A according to the reception and output program 60 executed in the RAM 48.

Next, description follows regarding the specific processing by the specific processing unit 290 of the data processing device 12. The units of the system described below are implemented by the data processing device 12 and the robot 414. In the following description the data processing device 12 is called a “server”, and the robot 414 is called a “terminal”.

### Example 1

Explanation of flow will be omitted due to being similar to a flow of the specific processing in Example 1 as described in the first exemplary embodiment above.

### Application Example 1

Explanation of flow will be omitted due to being similar to a flow of the specific processing in Application Example 1 as described in the first exemplary embodiment above.

### Example 2

Explanation of flow will be omitted due to being similar to a flow of the specific processing in Example 2 as described in the first exemplary embodiment above.

### Application Example 2

Explanation of flow will be omitted due to being similar to a flow of the specific processing in Application Example 2 as described in the first exemplary embodiment above.

The specific processing unit 290 transmits a result of the specific processing to the robot 414. In the robot 414, the control unit 46A outputs the result of the specific processing to the speaker 240 and the control target 443. The microphone 238 acquires audio representing user input in response to the specific processing result. The control unit 46A transmits audio data representing the user input as acquired by the microphone 238 to the data processing device 12. The specific processing unit 290 in the data processing device 12 acquires the audio data.

The data generation model 58 is a so-called generative artificial intelligence (AI). Examples of the data generation model 58 include generative AIs such as ChatGPT (registered trademark) (Internet search <URL: https://openai.com/blog/chatgpt>) and the like. The data generation model 58 is obtained by performing deep learning with a neural network. The data generation model 58 is input with a prompt including an instruction, and is input with inference data such as audio data representing speech, text data representing text, image data representing images (for example, still image data or video data), and the like. The data generation model 58 takes the input inference data, performs inference according to the instruction indicated in the prompt, and outputs an inference result in one or more data format from out of audio data, text data, image data, or the like. The data generation model 58 includes, for example, a text generative AI, an image generative AI, a multimodal generative AI, or the like. Reference here to inference indicates, for example, analysis, classification, prediction, and/or abstraction etc. The specific processing unit 290 performs the specific processing referred to above while using the data generation model 58. The data generation model 58 may be a model fine-tuned so as to output an inference result from a prompt not including an instruction, and in such cases the data generation model 58 is able to output an inference result from the prompt not including an instruction. There are plural types of the data generation model 58 included in the data processing device 12 or the like, and the data generation models 58 include an AI other than a generative AI. An AI other than a generative AI is, for example, a linear regression, a logistic regression, a decision tree, a random forest, a support vector machine (SVM), a k-means clustering, a convolutional neural network (CNN), a recurrent neural network (RNN), a generative adversarial network (GAN), a naïve Bayes, or the like and is capable of performing various processing, however there is no limitation to such examples. The AI may be an AI agent. Moreover, when the processing of each of the units mentioned above is performed by an AI, this processing is partly or entirely performed by the AI, however there is no limitation to such examples. Moreover, processing executed by an AI including a generative AI may be switched to rule-based processing, and rule-based processing may be switched to processing executed by an AI including a generative AI.

Although the processing by the data processing system 10 described above is executed by the specific processing unit 290 of the data processing device 12 or by the control unit 46A of the robot 414, the processing may be executed by a specific processing unit 290 of the data processing device 12 and a control unit 46A of the robot 414. Moreover, the specific processing unit 290 of the data processing device 12 acquires and collects information needed for processing from the robot 414 or from an external device or the like, and the robot 414 acquires and collects information needed for processing from the data processing device 12 or from an external device or the like.

For example, the collection unit is implemented by the control unit 46A of the robot 414 and/or by the specific processing unit 290 of the data processing device 12. For example, an acquisition unit acquires number-of-steps data using the camera 42 and/or the communication I/F 44 of the robot 414, and the number-of-steps data is processed by the specific processing unit 290 of the data processing device 12. For example, an analysis unit implemented by the specific processing unit 290 of the data processing device 12 analyzes data from the collection unit and the acquisition unit. For example, a generation unit implemented by the specific processing unit 290 of the data processing device 12 generates a cooking menu using a generative AI. For example, a supply unit implemented by the speaker 240 and the control target 443 of the robot 414 and/or the specific processing unit 290 of the data processing device 12 supplies the generated cooking menu to the user. Correspondence relationships of each unit to devices and control units are not limited to the examples described above, and various modifications thereof are possible.

The above exemplary embodiment gives an implementation example in which the specific processing is performed by the data processing device 12, however technology disclosed herein is not limited thereto, and the specific processing may be performed by the robot 414.

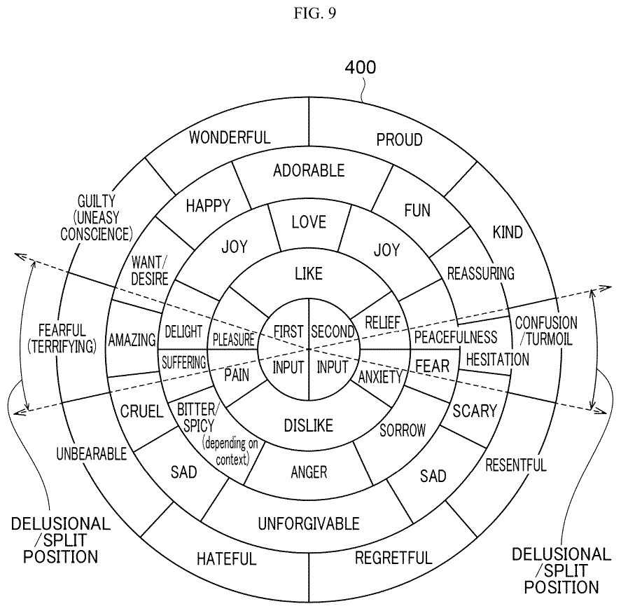

Note that the emotion identification model 59 serves as an emotion engine, and may decide the emotion of a user according to a specific mapping. Specifically, the emotion identification model 59 may decide the emotion of a user according to an emotion map (see FIG. 9) that is a specific mapping. Moreover, the emotion identification model 59 may also decide the emotion of the robot similarly, and the specific processing unit 290 may be configured so as to perform the specific processing using the emotion of the robot.

FIG. 9 is a diagram illustrating an emotion map 400 mapping plural emotions. In the emotion map 400, emotions are arranged in concentric circles that radiate out from the center. Primitive states of emotion are arranged nearer to the center of the concentric circles. Emotions expressing states and actions generated from states of mind are arranged further toward the outside of the concentric circles. Emotions are defined as including both affect and mental states. Emotions generated from reactions occurring in the brain are generally arranged at the left side of the concentric circles. Emotions induced by situational assessment are generally arranged at the right side of the concentric circles. Emotions generated from reactions occurring in the brain that are also emotions induced by situational assessment are generally arranged toward the top and toward the bottom of the concentric circles. Moreover, emotions of “euphoria” are arranged at the upper side of the concentric circles, and emotions of “dysphoria” are arranged at the lower side of the concentric circles. Plural emotions are accordingly mapped in this manner in the emotion map 400 based on a structure giving rise to emotions, and emotions that readily occur at the same time are mapped close to each other.

An example of such emotions is a distribution of emotions in the direction of 3 o'clock on the emotion map 400, generally around a boundary between relief and anxiety. Situational awareness dominates over internal sensations in the right half of the emotion map 400, with an impression of calm.

The inside of the emotion map 400 represents feelings, and the outside of the emotion map 400 represents actions, and so emotions further toward the outside of the emotion map 400 are more visible (are expressed by actions).

Human emotions are based on various balances, such as posture and blood sugar value balances, with a state of dysphoria being exhibited when these balances are far from ideal and a state of euphoria being exhibited when these balances are near to ideal. Even in a robot, a car, a motorbike, or the like, emotions can be thought of as being based on various balances such as orientation and remaining battery balances, with a state called dysphoria being exhibited when these balances are far from ideal and a state called euphoria being exhibited when these balances are near to ideal. An emotion map may, for example, be generated based on the emotion map of Dr. Mitsuyoshi (PhD Dissertation https://ci.nii.ac.jp/naid/500000375379: “Research on the phonetic recognition of feelings and a system for emotional physiological brain signal analysis”, Tokushima University). Emotions belonging to an area called “reaction” where feeling dominates are arranged in the left half of the emotion map. Moreover, emotions belonging to an area called “situation” where situational awareness dominates are arranged in the right half of the emotion map.

There are two types of emotion that facilitate leaning in an emotion map. One is an emotion in the vicinity of the center of negative “penitence” and “reflection” on the situational side. In other words, sometimes a negative “emotion” such as “I don't want to feel this way ever again” and “I don't want to be chided again” is experienced in a robot. Another is a positive emotion in the area of “desire” on the reaction side. In other words, there are times when a positive feeling such as “desire more” and “want to know more” is experienced.

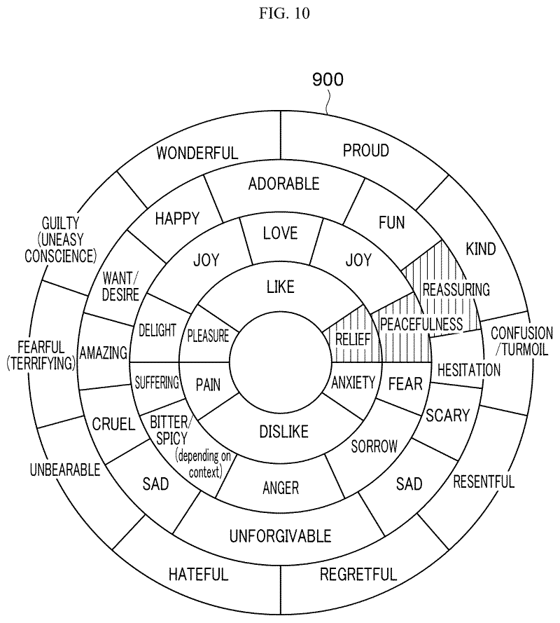

In the emotion identification model 59, user input is input to a pre-trained neural network, and emotion values indicating emotions shown on the emotion map 400 are acquired and the emotions of the user are decided. This neural network is pre-trained based on plural training data sets that each combine a user input with an emotion value indicating an emotion shown on the emotion map 400. The neural network is also trained such that emotions arranged close to each other have values that are close to each other, as in an emotion map 900 illustrated in FIG. 10. In FIG. 10 the plural emotions of “relief”, “peaceful”, and “reassured” are indicated as an example of close emotion values.

Although the system according to the present disclosure has been described mainly as functions of the data processing device 12, the system according to the present disclosure is not limited to being implemented in a server. The system according to the present disclosure may be implemented as a general information processing system. The present disclosure may, for example, be implemented by a software program operating on a personal computer, and may be implemented by an application operating on a smartphone or the like. The method according to the present disclosure may also be supplied to a user in the form of Software as a Service (SaaS).

Although in the exemplary embodiments described above examples are given of embodiments in which the specific processing is performed by a single computer 22, technology disclosed herein is not limited thereto, and distributed processing may be performed for the specific processing, with the specific processing distributed across plural computers including the computer 22. For example, the data generation model 58 may be provided in a device external to the data processing device 12, such that data generation in response to input data is performed in the external device.

Although in the exemplary embodiments described above examples are described of embodiments in which the specific processing program 56 is stored in the storage 32, the technology disclosed herein is not limited thereto. For example, the specific processing program 56 may be stored on a portable, non-transitory, computer readable, storage medium, such as universal serial bus (USB) memory or the like. The specific processing program 56 stored on the non-transitory storage medium is then installed on the computer 22 of the data processing device 12. The processor 28 then executes the specific processing according to the specific processing program 56.

Moreover, the specific processing program 56 may be stored on a storage device, such as a server connected to the data processing device 12 over the network 54, with the specific processing program 56 then being downloaded in response to a request from the data processing device 12 and installed on the computer 22.

Note that there is no need to store the entire specific processing program 56 on the storage device, such as a server connected to the data processing device 12 over the network 54, or to store the entire specific processing program 56 on the storage 32, and part of the specific processing program 56 may be stored thereon.

Hardware resources for executing the specific processing may use various processors as listed below. Examples of processors include, for example, a CPU that is a general-purpose processor that functions as a hardware resource to execute the specific processing by executing software, namely a program. Moreover, the processor may, for example, be a dedicated electronic circuit that is a processor having a circuit configuration custom designed for executing the specific processing, such as a field-programmable gate array (FPGA), a programmable logic device (PLD), or an application specific integrated circuit (ASIC). Memory is inbuilt or connected to each of these processors, and the specific processing is executed by each of these processors using the memory.

The hardware resource that executes the specific processing may be configured from one of these various processors, or may be configured from a combination of two or more processors of the same or different type (for example, a combination of plural FPGAs, or a combination of a CPU and a FPGA). The hardware resource executing the specific processing may be a single processor.

Examples of configurations of a single processor include, firstly, a configuration of a single processor resulting from combining one or more CPU and software, in an embodiment in which this processor functions as the hardware resource for executing the specific processing. Secondly, as typified by a System-on-chip (SOC) or the like, there is also an embodiment that uses a processor realized by a single IC chip to function as an overall system including plural hardware resources for executing the specific processing. Adopting such an approach means that the specific processing is realized using one or more of the various processors described above as hardware resource.

Furthermore, more specifically, an electrical circuit that combines circuit elements such as semiconductor elements or the like may be employed as a hardware structure of these various processors. The specific processing is merely an example thereof. This means that obviously redundant steps may be omitted, new steps may be added, and the processing sequence may be swapped around within a range not departing from the spirit of the present disclosure.

The described content and drawing content illustrated above are a detailed description of parts according to the present disclosure, and are merely examples of the present disclosure. For example, description related to the above configuration, function, operation, and advantageous effects is a description related to examples of the configuration, function, operation, and advantageous effects of parts according to the present disclosure. This means that obviously redundant parts may be eliminated, new elements may be added, and switching around may be performed on the described content and drawing content illustrated above within a range not departing from the spirit of the present disclosure. Moreover, to avoid misunderstanding and to facilitate understanding of parts according to the present disclosure, description related to common knowledge in the art and the like not particularly needing description to enable implementation of the present disclosure is omitted in the described content and drawing content illustrated as described above.

All publications, patent applications and technical standards mentioned in the present specification are incorporated by reference in the present specification to the same extent as if each individual publication, patent application, or technical standard was specifically and individually indicated to be incorporated by reference.

Note that, regarding the above description, the following supplementary notes are further disclosed.

### Example 1

### Supplementary 1

A system comprising a processor,
wherein the processor is configured to
control an information input and output interface to acquire condition information regarding a cooking target from a user,
read, from a storage device, past menu selection history information associated with the user and acquire the past menu selection history information together with the condition information,
analyze the condition information and the menu selection history information to generate a prompt sentence that instructs a generative artificial intelligence model to generate a menu and that defines an output format and constraint conditions,
transmit the prompt sentence to an external generative artificial intelligence model and acquire menu information generated by the generative artificial intelligence model based on the prompt sentence,
convert the acquired menu information into a predetermined data structure and transmit the converted menu information to a terminal device of the user so as to be presented via the information input and output interface, and
append and store the acquired menu information and a menu selection result by the user as the menu selection history information in the storage device, and reflect the menu selection history information in generation of a subsequent prompt sentence.

### Supplementary 2

The system according to supplementary 1,
wherein the processor is configured to
generate the prompt sentence by reflecting, in the prompt sentence, at least one of a health condition, a nutrition condition, and a cooking time condition included in the condition information, and by adding preference tendency information extracted from the menu selection history information, such that the prompt sentence instructs the generative artificial intelligence model to generate a health-oriented and personalized menu.

### Supplementary 3

The system according to supplementary 1,
wherein the processor is configured to
generate the prompt sentence by including, in the prompt sentence, constraint conditions relating to reduction of food waste and reduction of resource consumption, based on inventory ingredient information and use-by-date information of inventory ingredients included in the condition information, so as to instruct the generative artificial intelligence model to generate the menu, and update, in the storage device, a usage state of the inventory ingredients based on the acquired menu information so as to provide an economic benefit to the user.

### Application Example 1

### Supplementary 1

A system comprising a processor,
wherein the processor is configured to
receive condition information regarding a menu from a user via an information input/output unit,
acquire ingredient information in a storage apparatus by using a detection device,
generate a prompt sentence including the condition information and reflecting the ingredient information based on the acquired ingredient information and the condition information, and input the prompt sentence to a generative information processing model to cause the generative information processing model to execute a generation process of menu information,
analyze menu information output from the generative information processing model to extract required ingredient information for each menu by using a menu analysis unit, compare the required ingredient information extracted by the menu analysis unit with the ingredient information in the storage apparatus to calculate shortage ingredient information by using a shortage information calculation unit,
present, to the user, the menu information including the shortage ingredient information, and acquire menu selection information and order confirmation information from the user by using an order reception unit, and
store order information in a storage device based on the order confirmation information and transmit the order information to a delivery service device by using a delivery instruction unit.

### Supplementary 2

The system according to supplementary 1,
wherein the processor is configured to
structure health state information, subject attribute information, service time constraint information, and ingredient information in the storage apparatus included in the condition information, generate the prompt sentence including the structured information, and thereby instruct the generative information processing model to execute the generation process of the menu information that conforms to the health state information and satisfies the service time constraint information.

### Supplementary 3

The system according to supplementary 1,
wherein the processor is configured to
generate the order information such that economic benefit is provided to the user, by selecting the required ingredient information so as to reduce food waste amount based on the required ingredient information included in the menu information, the ingredient information in the storage apparatus, and expiration date information, and by determining the menu information so as to suppress power consumption amount based on operation information of the storage apparatus and the menu information.

### Example 2

### Supplementary 1

A system comprising a processor,
wherein the processor is configured to
acquire use conditions from a user through an information input/output device, the use conditions including at least one constraint related to utilization of stored items, and store the use conditions in a storage unit associated with the user,
acquire image data and physical quantity data from an imaging device and a detection device that are disposed in a storage apparatus, execute an image processing program on the image data to recognize a type of an item contained in the storage apparatus, estimate a quantity and a state of the item on the basis of the physical quantity data, and integrate a recognition result and an estimation result to generate inventory information,
analyze a transaction record image acquired by the user using a character recognition program, extract purchased item information from a text recognition result obtained by the character recognition program, and update the inventory information on the basis of the purchased item information,
generate a prompt sentence as an instruction sentence for input to a generative information processing model on the basis of the inventory information and the use conditions, and input the prompt sentence into the generative information processing model as a prompt to cause the generative information processing model to generate proposal information corresponding to the inventory information and the use conditions,
compare required items included in the proposal information with items included in the inventory information, identify insufficient items and substitutable items, and generate output data by adding a result of the identification to the proposal information, and
transmit the output data to the information input/output device and cause the information input/output device to present to the user the proposal information including information on the insufficient items and the substitutable items.

### Supplementary 2

The system according to supplementary 1,
wherein the processor is configured to
acquire, as part of the use conditions, at least one condition related to a health state of the user, reflect the at least one condition related to the health state in the prompt sentence, and thereby cause the generative information processing model to generate health-conscious proposal information.

### Supplementary 3

The system according to supplementary 1,
wherein the processor is configured to
control the prompt sentence and the proposal information on the basis of use-by date information and storage condition information included in the inventory information so as to reduce a quantity of discarded food and optimize energy consumption of the storage apparatus, and thereby provide an economic benefit to the user.

### Application Example 2

### Supplementary 1

A system comprising a processor,
wherein the processor is configured to
receive meal condition information and preference information from a user via an information input/output device,
acquire article information in a storage apparatus from a detection device including an imaging device and a measuring device, and execute image recognition processing and quantity estimation processing on the article information to generate a list of available articles,
extract additional purchase article information from a voucher image by character recognition processing and integrate the additional purchase article information into the list of available articles so as to update inventory information,
acquire emotion information indicating an emotional state of the user from an emotion estimation processing device and store the emotion information as user context,
generate a prompt sentence based on the inventory information, the meal condition information, the preference information, and the emotion information according to a template, and transmit the prompt sentence to a generative artificial intelligence model so as to instruct the generative artificial intelligence model to generate a menu or a meal plan,
analyze menu information obtained from the generative artificial intelligence model, extract required article information from the menu information, and specify shortage article information by comparing the required article information with the inventory information, present the menu information and the shortage article information to the user and, based on the shortage article information, generate and transmit provision request data for articles via an external service interface, and
perform message generation processing that changes a writing style or an expression tone of a notification sentence regarding the shortage article information and the menu information in accordance with the emotion information.

### Supplementary 2

The system according to supplementary 1,
wherein the processor is configured to generate, in the generation of the prompt sentence, a prompt sentence including a description that instructs the generative artificial intelligence model to generate a health-oriented menu or meal plan by reflecting a nutrition condition and an intake amount condition included in the meal condition information in the template.

### Supplementary 3

The system according to supplementary 1,
wherein the processor is configured to add, to the prompt sentence, a condition indicating articles to be preferentially used and a condition relating to cooking timing, based on expiration information and storage state information included in the inventory information, and to cause the generative artificial intelligence model to generate a menu that preferentially uses articles approaching expiration and contributes to reduction of energy consumption, thereby reducing a quantity of discarded food and optimizing resource consumption.

What is claimed is:

1. A system comprising:
circuitry configured to:
receive condition information from a user via an information input/output unit;
acquire ingredient information from a storage apparatus using a detection device;
structure health state information, subject attribute information, service time constraint information, and the ingredient information included in the condition information into a structured feature representation;
generate a prompt sentence incorporating the structured feature representation and the condition information;
input the prompt sentence to a generative information processing model to cause the generative information processing model to execute a generation process and output menu information;
analyze the menu information to extract required ingredient information for each menu item;
compare the required ingredient information with the ingredient information from the storage apparatus to calculate shortage ingredient information; and
present the menu information comprising the shortage ingredient information to the user and acquire menu selection information from the user.

2. The system of claim 1, wherein the circuitry is configured to:
generate the prompt sentence such that the generative information processing model generates menu information conforming to the health state information and satisfying the service time constraint information.

3. The system of claim 2, wherein the circuitry is configured to:
apply a nutritional analysis algorithm to the health state information and the subject attribute information to compute a set of nutritional constraint parameters; and
incorporate the nutritional constraint parameters into the prompt sentence as structured constraint fields that instruct the generative information processing model to generate menu items satisfying predefined nutritional criteria derived from the nutritional constraint parameters.

4. The system of claim 3, wherein the nutritional constraint parameters comprise at least one of a caloric intake range, a macronutrient ratio constraint, and a dietary restriction indicator derived from the health state information and the subject attribute information.

5. The system of claim 4, wherein the circuitry is configured to:
apply an emotion recognition algorithm to input information from the user to determine an emotional state; and
adjust at least one of a menu diversity parameter and a comfort food preference weight in the prompt sentence based on the determined emotional state.

6. The system of claim 1, wherein the detection device acquires the ingredient information by applying at least one of an image capture algorithm and a sensor reading algorithm to detect presence, quantity, and expiration date information of items stored in the storage apparatus.

7. The system of claim 6, wherein acquiring the ingredient information comprises applying an object detection algorithm to image data captured by the detection device to identify ingredient items and estimate their quantities, and reading expiration date data from the detected ingredient items.

8. The system of claim 7, wherein the object detection algorithm applies a convolutional neural network trained on a dataset of ingredient images to classify detected objects into ingredient category labels and estimate bounding box coordinates and quantity values.

9. The system of claim 1, wherein generating the prompt sentence comprises serializing the structured feature representation into a structured prompt template that encodes the health state information, the subject attribute information, the service time constraint information, the ingredient information, and an output format constraint specifying that the generative information processing model is to generate menu information in a machine-parseable format.

10. The system of claim 9, wherein the output format constraint specifies that the generative information processing model is to associate each generated menu item with a list of required ingredient identifiers and estimated quantities, enabling extraction of the required ingredient information by parsing the machine-parseable output.

11. The system of claim 1, wherein calculating the shortage ingredient information comprises computing, for each required ingredient identifier extracted from the menu information, a difference between a required quantity and an available quantity derived from the ingredient information, and generating a shortage record for each ingredient where the difference is positive.

12. The system of claim 11, wherein calculating the shortage ingredient information further comprises applying an expiration date filtering algorithm that excludes ingredient items from the ingredient information where the expiration date information indicates an expiration event prior to the service time indicated by the service time constraint information.

13. The system of claim 1, wherein the circuitry is configured to:
generate order information based on the shortage ingredient information and expiration date information by selecting shortage items so as to reduce a food waste amount derived from comparison of expiration dates and required quantities; and
determine menu items so as to suppress a power consumption amount based on operation information of the storage apparatus and the menu information.

14. The system of claim 13, wherein selecting shortage items to reduce the food waste amount comprises applying an optimization algorithm that minimizes a cost function comprising at least a weighted sum of predicted food waste quantity and total order cost subject to nutritional constraint parameters.

15. The system of claim 1, wherein the generative information processing model is a large language model accessed via a network interface, and wherein inputting the prompt sentence comprises transmitting a prompt request payload via an application programming interface.

16. The system of claim 6, wherein the detection device further acquires operation information of the storage apparatus comprising at least one of a current temperature setting, a compressor duty cycle, and an energy consumption metric.

17. The system of claim 16, wherein the circuitry is configured to:
incorporate the operation information into the structured feature representation such that the generative information processing model generates menu information that accounts for energy efficiency of the storage apparatus.

18. A system comprising:
circuitry configured to:
acquire ingredient information from a storage apparatus using a detection device;
receive condition information from a user comprising health state information, subject attribute information, and service time constraint information;
structure the health state information, the subject attribute information, the service time constraint information, and the ingredient information into a structured feature representation;
generate a prompt sentence incorporating the structured feature representation and input the prompt sentence to a generative information processing model to obtain menu information;
analyze the menu information to extract required ingredient information and compare the required ingredient information with the ingredient information to calculate shortage ingredient information; and
apply a nutritional analysis algorithm to the health state information and the subject attribute information to compute nutritional constraint parameters and incorporate the nutritional constraint parameters into the prompt sentence as structured constraint fields.

19. The system of claim 18, wherein the circuitry is configured to apply a convolutional neural network to image data captured by the detection device to classify detected objects into ingredient category labels and estimate bounding box coordinates and quantity values, and to apply an expiration date filtering algorithm that excludes ingredient items where the expiration date information indicates an expiration event prior to the service time indicated by the service time constraint information.

20. A method performed by circuitry, the method comprising:
receiving condition information from a user comprising health state information, subject attribute information, and service time constraint information;
acquiring ingredient information from a storage apparatus using a detection device;
structuring the health state information, the subject attribute information, the service time constraint information, and the ingredient information into a structured feature representation;
generating a prompt sentence incorporating the structured feature representation and the condition information;
inputting the prompt sentence to a generative information processing model to obtain menu information;
analyzing the menu information to extract required ingredient information;
comparing the required ingredient information with the ingredient information to calculate shortage ingredient information;
applying a nutritional analysis algorithm to compute nutritional constraint parameters from the health state information and the subject attribute information and incorporating the nutritional constraint parameters into the prompt sentence; and
presenting the menu information comprising the shortage ingredient information to the user.
